(19) United States
(12) Patent Application Publication
(10) Pub. No.: US 2026/0311822 A1
(43) Pub. Date: Oct. 8, 2026

(54) METHODS FOR MONITORING ENVIRONMENTAL IMPACT OF OUTPUT MATERIALS

(71) Applicant: BASF SE, Ludwigshafen am Rhein (DE)

(72) Inventors: Bastian GRUMBRECHT, Ludwigshafen am Rhein (DE); Claus TEUBER, Ludwigshafen am Rhein (DE); Olaf HUBER, Ludwigshafen am Rhein (DE)

(21) Appl. No.: 19/485,178

(22) PCT Filed: May 15, 2024
(86) PCT No.: PCT/IB2024/054686

§ 371 Date: Nov. 17, 2025

(30) Foreign Application Priority Data
May 17, 2023 (EP) 23173871.7
May 17, 2023 (EP) 23173878.2

(51) Int. Cl. G16C 60/00; G06F 16/901; G16C 20/10; G16C 20/30

(52) U.S. Cl. CPC G16C 60/00; G06F 16/9024; G16C 20/10; G16C 20/30

(57) ABSTRACT

The invention relates to the field of sustainability in particular environmental impact monitoring of output materials produced by chemical production networks. The invention relates to methods for monitoring an environmental property of an output material produced by a chemical production network, output materials associated with the environmental property and uses of the environmental property.

### TECHNICAL FIELD

The invention relates to the field of sustainability in particular environmental impact monitoring of output materials produced by chemical production networks. The invention relates to methods for monitoring an environmental property of an output material produced by a chemical production network, output materials associated with the environmental property and uses of the environmental property.

### TECHNICAL BACKGROUND

Chemical production networks are large scale production with multiple interconnected chemical production processes. To reduce resource usage such networks are highly integrated and produce multiple thousands of chemical end products. As a result, it is difficult to monitor such networks and particularly the associated resource usage.

### SUMMARY OF INVENTION

In one aspect disclosed is a method for monitoring an environmental property of an output material produced by a chemical production network, wherein the chemical production network includes multiple chemical processes for producing one or more output material(s) from one or more input material(s), the method comprising:
providing a product-based representation of the chemical production network adapted for allocating one or more environmental property contribution(s) associated with the production of one or more output material(s) through multiple interconnected chemical processes to the environmental property contributions per output material(s);
providing one or more environmental property contribution(s) related to the production of one or more output material(s) through multiple interconnected chemical processes;
generating the environmental property contributions per output material(s) by mapping the one or more environmental property contribution(s) related to the production of one or more output material(s) through multiple interconnected chemical processes according to the product-based representation of the chemical production network;
providing the environmental property contributions to the output material.

In another aspect disclosed is an apparatus for monitoring an environmental property of an output material produced by a chemical production network, wherein the chemical production network includes multiple chemical processes for producing one or more output material(s) from one or more input material(s), the apparatus comprising:
a provisioning interface configured to provide a product-based representation of the chemical production network adapted for allocating one or more environmental property contribution(s) associated with the production of one or more output material(s) through multiple interconnected chemical processes to the environmental property contributions per output material(s) and to provide one or more environmental property contribution(s) related to the production of one or more output material(s) through multiple interconnected chemical processes;
a contributions generator configured to generate the environmental property contributions per output material(s) by mapping the one or more environmental property contribution(s) related to the production of one or more output material(s) through multiple interconnected chemical processes according to the product-based representation of the chemical production network;
a data provider configured to provide the environmental property contributions to the output material.

In another aspect disclosed is an output material produced by the chemical production network, wherein the environmental property of the output material is provided according to the methods or by the apparatuses disclosed herein.

In another aspect disclosed is the use of the environmental property of the output material provided by the methods or by the apparatuses disclosed herein for determining an environmental property of a product produced from the output material.

### EMBODIMENTS

Chemical production networks are large scale productions producing more than thousands of chemical products through chemical conversion. To reduce the environmental impact of operating such networks, chemical production networks experience dynamic changes. This in particular concerns the input materials and chemical processes used in such chemical production networks. Since the chemical production networks are integrated chemical production networks with interconnected production chains and include multiple types of production processes for producing multiple output material(s) from multiple input material(s), monitoring resource usage, such as material flows and/or environmental impact, that is associated with the impact of the input materials or chemical processes on the environmental performance of the chemical products produced by the chemical production network is challenging.

By generating the environmental property contributions to the output material through mapping the one or more environmental property contributions related to the chemical production network according to the product-based representation of the chemical production network, the environmental impact can be reliably monitored per output material. In particular the product-based representation allows for monitoring the chemical production network and its environmental impact in a product or output material based logic. In other words, the environmental contributions may be mapped based on how many kg of a certain upstream output material needs to be produced or purchased in order to produce one kg of the end product. In yet other words, the environmental property contributions of the input materials provided to the chemical production network (raw material), the output material from pre-stages used as input material to a following stage or the chemical process of pre-stages may be mapped to discern the environmental contributions per end product. This enables monitoring of environmental property contributions by chemical processes and input materials used for producing the output material or end product by end product. Hence product-based monitoring can be conducted, which gives full transparency per end product leaving the chemical production network. This is particularly relevant for monitoring the environmental impact of the chemical production network to its produced end products. In chemical processes a plurality of intermediate materials may be produced, which are used for building up different end products. It is hence desirable to know the breakdown of the total environmental property for the end product per component involved in the production of the end product. The more stages are involved in the production of the end product the more complex this breakdown and/or mapping becomes. The disclosed methods, apparatuses, uses and output materials enable finding a distribution key in the form of a digital twin to allocate the contribution of each component involved in the production of the output materials to the share that is consumed for the output material of interest. It therefore may be seen as a principle of the methods to determine the share to the total environmental property caused by the production of the end product broken down to the component level.

In the following, embodiments of the present disclosure will be outlined by ways of examples. It is to be understood that the present disclosure is not limited to said embodiments and/or examples.

The environmental property may relate to greenhouse gas emissions, carbon emissions or product carbon footprint. The environmental property of the output material produced by the chemical production network may relate to environmental property contribution(s) associated with the production of the output material. The environmental property contribution(s) may relate to different contribution types associated with different sources for the contributions. Sources may be the input materials used to produce the output material, the chemical processes used to produce the output material, transport of output materials used as input materials to produce other output materials inside the chemical production network and/or energy inputs used to produce the output material. For greenhouse gas emissions, carbon emissions or carbon footprints, contributions may be clustered by scope 1, 2 or 3 contributions as defined the Greenhouse Gas Protocol Standard or the European Commission Product Environmental Footprint (PEF 2021). The environmental property contributions may be aggregated to one environmental property. The environmental property may include contributions from different contribution types. The environmental property may specify contributions for different contribution type. For example, the environmental property may specify the carbon footprint or product carbon footprint of the output material. Further for example, the environmental property may specify the carbon footprint or product carbon footprint for scope 1, 2 and 3 aggregated, summed or separately.

One or more environmental property contributions related to the production of one or more output material(s) may relate to contributions of production components used by the chemical production network. Production components may include input materials, chemical processes and/or transports. One or more environmental property contributions related to the production of one or more output material(s) may relate to input materials, chemical processes and/or transports used to produce the output material. The contributions related to chemical process may include contributions directly or indirectly related to the chemical process. For example, external energy inputs to chemical processes such as electricity may be indirectly related to the chemical processes. Further for example, internal energy inputs to chemical processes such as steam or heat may be directly related to the chemical processes.

Chemical production networks may include multiple types of production processes for producing different output materials from input materials. The chemical production network may include a production network producing multiple output materials in multiple production chains. The chemical production network may include connected, interconnected and/or non-connected production chains or chemical processes. The chemical production network may produce from input materials multiple intermediates and from intermediates multiple end products. The output material may be an intermediate material used in a different chemical process as input material and/or an end product produced by multiple chemical processes or at least partially interconnected chemical processes. Multiple chemical processes may be connected and/or interconnected to produce the output material(s), e.g. end product(s), of the chemical production network. Multiple chemical processes connected and/or interconnected to produce the output material(s), e.g. the end product(s), may form a production chain, value chain and/or production path.

The chemical production network may comprise one or more entry point(s) at which input materials are provided to the chemical production network. Input material may enter the chemical production network at entry points. The chemical production network may comprise one or more exit point(s) at which output materials are provided from the chemical production network. End products may be output materials leaving the production network at exit points. The system boundary of the chemical production network may be defined by the entry and exit points. The system boundary of the chemical production network may be defined by the input materials provided to the chemical production network, the chemical process converting the input materials to output materials and the end products as provided by the chemical production network and produced via the chemical processes, e.g. connected or interconnected chemical processes.

The chemical production network may include multiple chemical processes for producing one or more output material(s) from one or more input material(s). The chemical process may convert one or more input material(s) to one or more intermediate material(s). The chemical process may convert one or more intermediate material(s) to one or more end product(s). The chemical process may convert one or more input material(s) to one or more output material(s). The chemical process may chemically, physically, mechanically and/or thermally convert one or more input material(s) to one or more output material(s). The chemical process may be associated with a multi-input-multi-output relation related to the input material(s) provided to the chemical process and the output material(s) produced by the chemical process. One or more chemical process(es) may form a sub-cluster or a plant of the chemical production network. E.g. multiple chemical processes may form the sub-cluster or the plant for producing one or more output material(s) from one or more input material(s) provided to the sub-cluster or plant.

The chemical production network may include one or more sub-cluster(s). A sub-cluster may include one or more chemical process(es). The sub-cluster may include one or more chemical plant(s). The sub-cluster may include multiple connected and/or interconnected chemical process(es) and/or plant(s). The sub-cluster may produce based on one or more input material(s) e.g. provided to the sub-cluster, one or more output material(s). Multiple sub-clusters may be connected and/or interconnected.

The representation may be a digital representation or digital twin of the chemical production network. The network-based representation of the chemical production network may map the chemical production network based on production data and production network data to one or more material input-output relations per chemical process. For generating the network-based representation production data and production network data may be merged. The network-based representation may include input-output relations per chemical process as may be determined from production data and production network data. The input-output relations may specify the input materials per chemical process, the input material share per chemical process, the origin classification per input material, the input material share per origin classification, the output materials per chemical process, the output material share per chemical process and/or the output material type per chemical process. The share may refer to a quantity or quantity ratio.

Chemical production networks may include material flows with cycles or material may be recirculated. Cycles may include material flows, in which output material is fed back into at least one preceding chemical process or any chemical process connected to at least one preceding chemical process. The preceding or prior chemical process may refer to any chemical process that produces output material which is directly or indirectly used to produce the input material of any subsequent chemical process. Cycles may relate to recirculation of output material of a first chemical process as input material to a second chemical process. The second chemical process may be any preceding or prior chemical process. Material recirculation may relate to one chemical process in which at least one of the output materials is recircled as input material to the chemical process. Material recirculation may relate to one chemical process in which at least one of the output materials is recircled as input material to the chemical process. Material recirculation may stretch over one or more chemical processes of the chemical production network. Material recirculation may relate to a chain of multiple chemical processes in which at least one of the output materials of at least one e.g. the last chemical process, of the chain of multiple chemical processes is recircled as input material to any preceding chemical process, e.g. the first chemical process, of the chain of multiple chemical processes.

The network-based representation may represent the material flows based on the chemical processes of the chemical production network. The network-based representation may follow a network logic based on chemical processes connected through material flow(s) of the chemical production network. The network-based representation may re-fleet the material flow of the chemical production network by way of the chemical processes using and/or producing the materials, such as input and/or output materials.

The product-based representation may provide product relations as determined from the input-output relations of the network-based representation to map the resource usage for producing the products of the chemical production network. The product-based representation may include or be an output material, e.g. intermediate or end-product, based representation. Product may include output material, e.g. any intermediate or end-product. The output material-based representation may provide output material relations as determined from the input-output relations of the network-based representation to map the resource usage for producing the output materials of the chemical production network. One example of the output material may be an intermediate product or an end product. The end product-based representation may provide end-product relations as determined from the input-output relations of the network-based representation to map the resource usage for producing the end products of the chemical production network. For generation of the product based representation the network-based representation may be generated and transformed. The product or output material, preferably end-product, based representation may include product or output material, preferably end-product, relations per product or output material, preferably end-product, of the chemical production network. The product or output material, preferably end-product, relation may relate to the production chain per product or output material, preferably end product. The product or output material, preferably end-product, relation may relate to or include a quantity, a quantity share and/or a quantity ratio per input material, inter-mediate material, output material and/or chemical process(es) used to produce the product or output material, prefer-ably end product. The chemical processes and associated material flows may be connected via the production chain of the product or output material, preferably end-product. The product or output material, preferably end-product, relation may relate to or include the quantity, the quantity share and/or the quantity ratio per input material and/or inter-mediate material in relation to a unit quantity of output material, e.g end product, e.g. per 1 kg output material or end product.

The network-based representation may provide a network-based attribution matrix. The product-based representation may provide a product-based attribution matrix or the output material-based representation may provide a output material-based attribution matrix.

The output material, preferably end-product, based representation may represent the material flows based on the output material, preferably end-product, produced by chemical processes of the chemical production network. The output material, preferably end-product, based representation may follow a product logic based on materials and chemical processes used to produce the output material, preferably end product. The output material, preferably end-product, based representation may reflect the material flow of the chemical production network by way of a contribution of chemical processes and associated material connected to produce the output material, preferably end product.

In one embodiment providing a product-based representation of the chemical production network includes
gathering production data associated with the chemical processes and material flows from input material(s) to output material(s) per chemical process,
gathering production network data associated with material flows to the chemical production network and/or between chemical processes of the chemical production network,
generating a product-based representation of the chemical production network based on the production data and the production network data.

In another embodiment the network-based representation may be generated that maps the chemical production network based on the production data and the production network data to one or more material input-output relations per chemical process. The network-based representation may be transformed to the output material, preferably end product, based representation by determining output material, preferably end-product, relations from the input-output relations for mapping or to map the resource usage for producing the output materials, preferably end products, of the chemical production network. The output material or product-based representation of the chemical production network may be provided.

In another embodiment generating the network-based and/or production-based representation includes removing and/or resolving one or more material recirculation(s) within chemical process(es) or per chemical process, in which at least one of the output materials of the chemical process is recircled as input material to said chemical process, and/or one or more material recirculation(s) relating to a chain of multiple chemical processes in which at least one of the output materials of at least one chemical process of the chain of multiple chemical processes is recircled as input material to any preceding chemical process of the chain of multiple chemical processes. For generating the network-based and/or production-based representation one or more material recirculation(s) within chemical process(es), in which at least one of the output materials of the chemical process is recircled as input material to said chemical process, may be resolved and/or removed. For generating the network-based and/or production-based representation one or more material recirculation(s) relating to a chain of multiple chemical processes in which at least one of the output materials of at least one, e.g. the last chemical process, of the chain of multiple chemical processes is recircled as input material to any preceding chemical process, e.g. the first chemical process, of the chain of multiple chemical processes may be resolved and/or removed. In other words, the network-based and/or the product-based representation may represent the material flows, wherein cycles or recirculation per chemical process or per chain of chemical processes are resolved and/or removed.

The production data or network-based representation generated based on the merged production data and production network data may represent the material flows based on the chemical processes of the chemical production network including one or more material cycle(s) or recirculation(s). The material recirculation within chemical process(es), in which at least one of the output materials of the chemical process is recircled as input material to said chemical process, may be resolved and/or removed from the production data or the network-based representation generated based on the merged production data and production network data. In other words, the network-based representation may represent the material flows, wherein cycles or recirculation per chemical process are resolved and/or removed. Recirculations and/or cycles associated with recirculation within chemical processes or in other words recirculation in one chemical process may be resolved and/or removed from the production data or the network-based representation. The cycles may be identified based on chemical process identifier(s) related to the same input and output material for the chemical process. Such relation may be identified based on the material type(s), e.g input, output, by, side or the like, per chemical process identifier that may indicate the same material on input and output side of the chemical process. To resolve and/or remove the cycle(s) per chemical process from the production data or the chemical process recipes of the production data, the quantities associated with respective material may be transformed to a net quantity relating to the material type with the larger quantity. In yet other words, the network-based representation may represent the material flows including cycles or recirculation across at least one chain of multiple chemical processes.

The removing and/or resolving of one or more material recirculation(s) relating to a chain of multiple chemical processes may include transforming the network-based and/or production-based representation to a graph data structure. The network-based and/or production-based representation may be transformed to remove and/or resolve one or more material recirculation(s) relating to a chain of multiple chemical processes in which at least one of the output materials of at least one, e.g. the last chemical process, of the chain of multiple chemical processes is recircled as input material to any preceding chemical process, e.g. the first chemical process, of the chain of multiple chemical processes. At least part of the network-based and/or production-based representation may be transformed to a graph data structure associated with chemical processes as vertices and material flows as edges. The graph data structure may include a directed graph representation including the direction of material flow, such as input material flowing to the chemical process or output material flowing from the chemical process. The graph data structure may be at least partially used to transform the network-based representation to the product-based representation. The graph representation may be transformed to combine vertices and edges that form at least one cycle or material recirculation relating to a chain of multiple chemical processes in which at least one of the output materials of at least one, e.g. the last chemical process, of the chain of multiple chemical processes is recircled as input material to any preceding chemical process, e.g. the first chemical process, of the chain of multiple chemical processes. In particular, the part of the graph representation associated with material recirculation(s) relating to a chain of multiple chemical processes in which at least one of the output materials of at least one, e.g. the last chemical process, of the chain of multiple chemical processes is recircled as input material to any preceding chemical process, e.g. the first chemical process, of the chain of multiple chemical processes may be transformed into the graph data structure. The trans-formed and/or directed graph representation may be topologically ordered. The ordered graph representation may be used to generate the product-based representation of the chemical production network. By using the graph data structure at least for those parts of the network-based representation, which are associated with recirculation of material across chemical processes, the recirculation(s) can be eliminated through graph transformation aggregating the nodes and edges associated with recirculation in one or more node(s) of the graph data structure.

Production data may be associated with the chemical processes and material flows from input material(s) to output material(s) per chemical process. Production data may include one or more multi-input-multi-output relation(s) associated with chemical processes of the chemical production network. The multi-input-multi-output relation may relate to the input material(s) provided to the chemical process and the output material(s) produced by the chemical process. The production data may include input material quantity and output material quantity per material per chemical process. Production data may include measurement data provided by the chemical production network per chemical process. The production data may include measured time series data for input material quantity and output material quantity per material per chemical process. Production data may include measurement data provided by the chemical production network per chemical process.

Production network data may be associated with material flows to the chemical production network and/or between chemical processes, preferably sub-clusters, of the chemical production network. Production network data may include one or more input material relation(s) related to an origin classification of the input material. Production network data may include per input material one or more origin classification(s) and/or respective shares per origin classification. Production network data may include per chemical process and/or sub-cluster and per input material one or more origin classification(s) and/or respective shares per origin classification. The shares per origin classification may relate to the input material quantity related to the origin classification. The quantity of input material may quantify the amount of input material provided to the chemical production network, the chemical process and/or the sub-cluster. The quantity of input material may relate to mass, volume or similar measures suitable for quantifying the amount of input material. Production network data may include the origin classification of the input material and production network ratios or consumption mixes signifying the quantity of input material per origin classification.

In another embodiment generating a product-based representation includes generating a network-based representation that maps the chemical production network based on the production data and the production network data to one or more material input-output relations per chemical process, transforming the network-based representation to a product-based representation by determining output material relations from the input-output relations for mapping the environmental property contributions related to the production of one or more output material(s) for mapping the environmental property contributions related from the production of one or more output material(s) to the environmental property contributions per output material(s). The network-based representation may be generated that maps the chemical production network based on the production data and the production network data to one or more material input-output relations per chemical process.

In another embodiment the network-based representation is adapted for allocating one or more environmental property contribution(s) associated with the production of one or more output material(s) according to a network flow logic. In other words, the one or more environmental property contribution(s) may be mapped by network component. The network flow logic may refer to a network component logic. Network components may include chemical processes, subclusters and/or material flows including input materials, output materials, intermediate materials, end products or any combination thereof. The input-output relations may provide for the mapping by network component. The input-output relations may relate to quantity, quantity share and/or quantity ratio by network component.

In another embodiment the product-based representation is adapted for allocating one or more environmental property contribution(s) associated with the production of one or more output material(s) according to a material use logic. In other words, the one or more environmental property contribution(s) may be mapped by output material production path. Output material production path may signify the production path through the chemical production network from input material to output material, e.g. end product. Output material production path may include chemical processes, subclusters and/or material flows including input materials, output materials and/or intermediate materials that contribute to the output material or end product of interest. The product or output material relations may provide for the mapping by output material production path. The product or output material relations may relate to quantity, quantity share and/or quantity ratio by output material production path.

In another embodiment at least part of the product-based representation is transformed to a graph data structure associated with chemical processes or subclusters as vertices and material flows as edges, wherein the graph data structure includes metadata associated with the environmental property contributions per output material, e.g. end product, production path, wherein the environmental property contributions per chemical process or subcluster are added to the vertices and the emission contributions per material flow are added to the edges. The graph data structure may be a directed graph data structure reflecting the material flows between chemical processes and/or the sequence of chemical processes. The vertices and edges including metadata associated with the environmental property contributions may be provided to a user interface e.g. in a sequential manner. Providing to the user interface may include displaying on a screen. At least two vertices and at least one edge including metadata associated with the environmental property contributions may be provided to the user interface. The vertices and edges including metadata associated with the environmental property contributions may be provided to an interactive user interface, which adapts the at least two vertices and at least one edge provided to the user interface upon user interaction. For example, the vertices and/or edge(s) may be expanded or collapse to provide more or less vertices and/or edge(s). The interaction may be based on the graph data structure and the vertices connected through edges. For example, vertices may be run through in sequential order. Further for example, multiple vertices may be collapsed to a summed vertex or a summed vertex may be expanded to multiple vertices including at least one edge between the vertices. E.g. chemical process vertices with associated material flow edges may by collapsed to subcluster vertex with associated material flow edges or a subcluster vertex may be expanded to chemical process vertices with associated material flow edges. In yet other embodiments the environmental property contributions per material including input material(s) and/or output material(s), such as intermediate material(s) and/or end-product(s), to the environmental property of the output material, e.g. end-product, of interest may be provided to the user interface. The environmental property contributions per material may include the summed environmental property per material of the production path. For example, the environmental property contribution related to the input material, the chemical process and/or transport of input materials and contributing to the environmental property contribution of the material may be added to provide the total environmental property contribution per material. This way the total environmental property contribution of input material produced by the chemical production network may be provided. Such providing of environmental property and/or environmental property contributions per material of the production path allows for more reliable and simpler monitoring by the operator of the chemical production network. Through the material use logic the output materials of the chemical production network may be monitored. This is particularly useful for mapping the environmental property contributions of the chemical production infrastructure to the environmental property of the output materials, e.g. end products.

In another embodiment a network-based representation of the chemical production network is provided. At least part of the network-based representation may be transformed to a graph data structure associated with chemical processes or subclusters as vertices and material flows as edges, wherein the graph data structure includes metadata associated with the environmental property contributions per network component, wherein the environmental property contributions per chemical process or subcluster are added to the vertices and the emission contributions per material flow are added to the edges. The graph data structure may be a directed graph data structure reflecting the material flows between chemical processes and/or the sequence of chemical processes. The vertices and edges including metadata associated with the environmental property contributions may be provided to a user interface as described herein, particularly in the context of the graph data structure for the product-based representation. In yet other embodiments the environmental property contributions per network component, e.g. chemical process, material flow and/or subclustered, may be provided to the user interface. The environmental property contributions per network component may include the summed environmental property per network component of the chemical production network. For example, the environmental property contributions related to the chemical process, e.g. including the environmental property contribution of input material(s) or energy inputs to the chemical process, may be added to provide the total environmental property contribution per chemical process. This way the total environmental property contribution of the chemical processes of the chemical production network may be provided. Such providing of environmental property and/or environmental property contributions per network component allows for more reliable and simpler monitoring by the operator of the chemical production network. Through the network component logic or network flow logic the infrastructure of the chemical production network may be monitored. This is particularly useful for monitoring the network components of the chemical production infrastructure making up the environmental properties of end products of the chemical production network.

In another embodiment the one or more environmental property contribution(s) relate at least to the one or more input material(s) provided to the chemical production network, the chemical processes of the chemical production network, and/or transport of input material(s) inside the chemical production network. The one or more environmental property contribution(s) associated with the production of one or more output material(s) may be provided per network component. The one or more environmental property contribution(s) associated with the production of one or more output material(s) may be provided for input material(s), chemical process(es) and/or transport(s) of input material(s) inside the chemical production network.

In another embodiment the one or more environmental property contribution(s) associated with the production of one or more output material(s) are provided per input material, per chemical process and/or per transport of input material(s) inside the chemical production network. The one or more environmental property contribution(s) associated with the production of one or more output material(s) may be provided for input materials entering the chemical production network, for activities directly related to chemical processes, such as the use of internal energy inputs, internal steam or the generation of waste water, for activities indirectly related to chemical processes, such as the use of external energy inputs, and/or for transport of materials inside the chemical production network, such as internal transport between chemical processes or subclusters of the chemical production network.

In another embodiment the environmental property contributions relate to carbon emissions of the chemical production network. The environmental property contribution(s) may relate at least to carbon emissions associated with the one or more input material(s) provided to the chemical production network, carbon emissions associated with the chemical processes of the chemical production network, carbon emissions associated with the transport of input material(s), and/or carbon emissions associated with energy usage of the chemical production network.

In another embodiment providing one or more environmental property contribution(s) related to the production of one or more output material(s) includes gathering emission data from distributed monitoring systems of the chemical production network. The emission data may relate to emissions generated on production of output materials by the chemical production network. The emission data may relate to measured emissions generated on production of output materials by the chemical production network. The emission data may relate to historic emission data gathered on production of output materials by the chemical production network. The emission data may be gathered over a time period of production of output materials by the chemical production network.

In another embodiment the emission data is provided per chemical process and/or per subcluster including one or more chemical process(es). The emission data may be at least partially provided per subcluster including one or more chemical process(es), wherein the emission data is at least partially allocated to chemical process(es) of the subcluster. The emission data signifying carbon emissions per subcluster may be allocated to at least one chemical process by production quantity, production share and/or production quantity ratio with respect to the subcluster.

In one embodiment the network-based representation may be generated that maps the chemical production network based on the production data and the production network data to one or more material input-output relations per chemical process. The network-based representation may be transformed to the output material, preferably end product, based representation by determining output material, preferably end-product, relations from the input-output relations for mapping or to map the resource usage for producing the output materials, preferably end products, of the chemical production network. The product-based representation of the chemical production network may be provided.

In another embodiment the production data are gathered per chemical process and/or the production network data are gathered for one or more chemical process(es) forming a subcluster of the chemical production network. The production data may be associated with chemical processes or the chemical production network. The production network data may be associated with subclusters of the chemical production network. The production data may include to input-output relations of chemical processes. The production data may relate to input material quantities and output material quantities per chemical process. The production data may relate to input material quantities per input material used by the chemical process. The production data may specify a chemical process identifier associated with the chemical process. The production data may relate to output material quantities per output material used by the chemical process. The production data may relate to or include input material quantities and output material quantities per chemical process identifier. The production data may relate to or include an output material type per output material of the chemical process. The output material type may be associated with a main output material, a side output material or waste. The output material type may specify the type of output material as produced by the chemical process producing multiple output materials, e.g. from multiple input materials. Production network data may be associated with input material quantities provided to the chemical production network and/or between sub-clusters. Production network data may include network ratios or consumption mixes specifying the share per input material origin. By providing production data per chemical process and production network data per subcluster, the input materials of the chemical processes can be further assigned the origin classification and repressive shares per origin classification. This way the representation of the chemical production network can discern between input materials provided to the chemical production network and input materials provided between chemical processes or sub-clusters within the chemical production network. Such distinction provides for more flexibility in monitoring the re-source usage, since the internal resource usage and the external resource usage can be separately monitored.

In another embodiment the production data include aggregated production measurement data per chemical process. The production measurement data may be aggregated over time. The production data may include quantity measurements for input material quantities as provided to the chemical process and/or output material quantities as produced by the chemical process. The measured quantities over time may be aggregated over one or more time interval(s). The measured quantities over time may be aggregated over one or more time interval(s) depending on the chemical process, the subcluster, the plant, and/or the production chain. Such aggregation may include an average over time or any other aggregation methods suitable to aggregate over time. By providing production data or chemical process recipes from measured data the monitoring of resource usage can be further enhanced, since the monitoring can be tailored to suitable time intervals. For example, in stable production situations with on average stable material quantities, the aggregation over time may be elongated to e.g. 1, 2 or 3 years. Further for example, in dynamic production situations with fluctuations in production quantities the material quantities can be more regularly updated. This way the update of measurement data and the respective aggregation may be tailored to the chemical process, the subcluster, the plant, and/or the production chain.

In another embodiment the production data include multi-input-multi-output relations per chemical process. The multi-input-multi-output relations may be associated with the input materials provided to the chemical process and the output materials produced by the chemical process. The multi-input-multi-output relations per chemical process may relate to or include quantities of input materials provided to the chemical process and quantities of output materials produced by the chemical process. The multi-input-multi-output relations per chemical process may be transformed to multi-input-single-output relations by using allocation rules that separate the output materials by mapping the re-quired input material quantities to respective output materials. Through the mapping to multi-input-single-output relations fork or loops in chemical process recipes can be eliminated enabling and simplifying processing of representation of the chemical production network. This way the production chain per output material or end product can be reliably constructed. This allows for more reliable monitoring of resource usage per output material or end product.

In another embodiment the input-output relations are determined by combining the production data and the production network data based on the input materials per chemical process and/or per subcluster including one or more chemical process(es). In another embodiment the production data and the production network data are merged by providing production network ratios and chemical process recipes and by gathering the production network ratios and chemical process recipes according to a material flow of the chemical production network. In another embodiment the production network ratios signify consumption mixes for input materials provided to the chemical production network and/or provided from one or more chemical process(es) to one or more subsequent chemical process(es), wherein the chemical process recipes signify input materials, output materials and interrelations between input materials and output materials.

Merging the production data including chemical process recipes and the production network data may include adding the network ratios or consumption mixes to the chemical process recipes. In particular the origin classification and the respective share per input material specified by the chemical process recipe may be added. This way the representation of the chemical production network can be extended to discern external and internal input material. The monitoring of the chemical production network can hence be enhanced to discern internal from external activities.

In another embodiment the network-based representation is generated based on an input-output model that relates the resource usage per material, e.g. input or output material, to one or more resource contribution(s) of the chemical processes and/or materials. The network-based representation may be generated based on production data and production network data. The network-based representation may be generated by providing per material, e.g. input material or output material, the network ratio(s) and/or the material quantity/ies per material. This way the input factors for different resource contributions from chemical processes and/or materials may be determined. The network-based representation may be generated and/or updated by further providing resource usage data including or related to resource contributions attributable to chemical processes and/or the materials. The resource contribution(s) of the chemical processes and/or materials may be provided by the resource usage data. The attribution may be provided by the production data and/or production network data, in particular the network ratio(s) per input material and/or the material quantity/ies per material. An indexed or structured matrix data structure may be generated based on the production data, production network data and/or resource contribution data. The indexed or structured matrix data structure may be generated per material according to a material flow logic where resource contributions of the chemical processes and/or material quantities may be gathered per material according to the input-output model. The indexed or structured matrix data structure may be a network-based attribution matrix.

In another embodiment at least part of the network-based representation is transformed to a graph data structure associated with chemical processes as vertices and material flows as edges. The graph data structure may be a directed graph representation including the direction of material flow, such as input material flowing to the chemical process or output material flowing from the chemical process. The graph data structure may be at least partially used to transform the network-based representation to the product-based representation. In another embodiment the graph representation is transformed to combine vertices and edges that form at least one cycle. In particular, the part of the network-based representation associated with recirculation of material from one chemical process to another chemical process or form one chemical process to a preceding chemical process may be transformed into the graph data structure. Recirculation may stretch over one or more chemical processes of the chemical production network. The transformed and/or directed graph representation may be topologically ordered. The ordered graph representation may be used to generate the product-based representation of the chemical production network. By using the graph data structure at least for those parts of the network-based representation, which are associated with recirculation of material across chemical processes, the recirculation(s) can be eliminated through graph transformation aggregating the nodes and edges associated with recirculation in one ore more node(s) of the graph data structure. The result of such transformation may be added to the indexed or structured matrix data structure. The indexed or structured matrix data structure or network-based attribution matrix may be transformed to an product or output material-based attribution matrix. The transformation of the network-based representation provided by the indexed or structured matrix data structure to the end-product based representation may thus be executed more efficiently requiring less compute time and resources.

In another embodiment the network-based representation is transformed from a network flow logic to a material use logic, wherein the network flow logic relates to material flows per chemical process of the chemical production network and the material use logic relates to the material flows per end product of the chemical production network. In another embodiment the product-based representation of the chemical production network is adapted to monitor resource usage of the chemical production network per end product. The network-based representation of the chemical production network may be adapted to monitor resource usage of the chemical production network per chemical process. To extend the network-based and/or end-product based representation are used to monitor resource usage, resource contribution data including resource contributions attributable to chemical processes and/or the materials may be provided.

### BRIEF DESCRIPTION OF THE DRAWINGS

The description of drawings is provided for illustrative purposes and shall not be considered limiting. The embodiments and examples are illustrative embodiments and examples to further line out the concepts lined out herein. The figures include schematic illustrations and shall not be considered limiting. Other embodiments and examples that fall under the concepts lined out herein are possible and may not be explicitly described herein.

FIGS. 1a-c illustrate examples of chemical processes with multi-input-multi output relations.

FIG. 2 illustrates a simplified schematic view of multiple chemical processes of the chemical production network.

FIG. 3 illustrates a simplified schematic view of a sub-cluster of the chemical production network including multiple chemical processes.

FIG. 4 Illustrates a simplified schematic view of multiple sub-clusters forming the chemical production network.

FIG. 5 illustrates an example flow chart of a method for generating digital representations of the chemical production network and/or updating the digital representation for monitoring the chemical production network.

FIG. 6 illustrates an example chemical process recipe, and an allocation rule as it may be applied to the example chemical process recipe.

FIG. 7 illustrates an example of connected chemical process recipes as provided by the production data and an example transformation for single output recipes.

FIG. 8 illustrates an example of production network data including origin classification and production network ratios.

FIG. 9 illustrates an example of a network-based representation of the chemical production network.

FIG. 10 illustrates an example of a network-based representation of the chemical production network in a graph data structure.

FIG. 11 illustrates an example of a network-based graph representation of the chemical production network as may be displayed on a user interface.

FIG. 12 illustrates an example of a model for generating the network-based representation.

FIG. 13 illustrates an example extract of the network-based representation as generated from fork-free or loop-free chemical process recipes.

FIG. 14 illustrates another example extract of the network-based representation as generated from fork-free or loop-free chemical process recipes.

FIG. 15 illustrates an extract or sub-structure of the network-based representation in a graph data structure.

FIG. 16 illustrates another extract or sub-structure of the network-based representation in a transformed graph data structure.

FIG. 17 illustrates another example of a model for generating the network-based representation.

FIG. 18 illustrates another example extract of the network-based representation including emission contributions.

FIG. 19 illustrates an example user interface generated based on the network-based and/or product-based representation.

FIG. 20 illustrates an example of a flowchart for monitoring emission contributions of chemical production network e.g. per output material such as per end product of the chemical production network.

FIG. 21 illustrates a schematic example of emission contributions per chemical process.

FIG. 22 illustrates a schematic example of emission contributions per end product.

FIG. 23 illustrates a schematic example of emission contributions in a value chain.

FIGS. 24-26 illustrate schematic examples of monitoring emission contributions in a value chain of the chemical production network based on input materials such as raw materials provided to the chemical production network, process emissions and/or production volumes.

### DETAILED DESCRIPTION

Chemical production networks include multi-input-multi-output chemical processes. This makes chemical production networks complex not only in the physical world, but also in their representation in digital systems that require a digital twin for monitoring such networks. One example for monitoring may be the monitoring of resource usage such as emissions or environmental impact of the chemical products produced by the chemical production network. To monitor chemical production networks with such complexity and scale, the following challenges exist:
1. Highly decentralized monitoring data stored in relation to different hierarchy levels of the chemical production network, such as chemical processes, plants or sub-clusters needs to be gathered to form a digital twin.
2. Generating a digital representation of such networks needs to take account of the multi-in multi-out nature of the chemical processes.
3. The computing resources required to generate the digital twin or perform operations on the digital twin are high generating further emissions due to the computing resources required.
4. Reliable monitoring requires mapping of monitoring data to the digital twin, in particular network-based monitoring data may be mapped to product-based logic or emission data may be mapped to the digital twin.

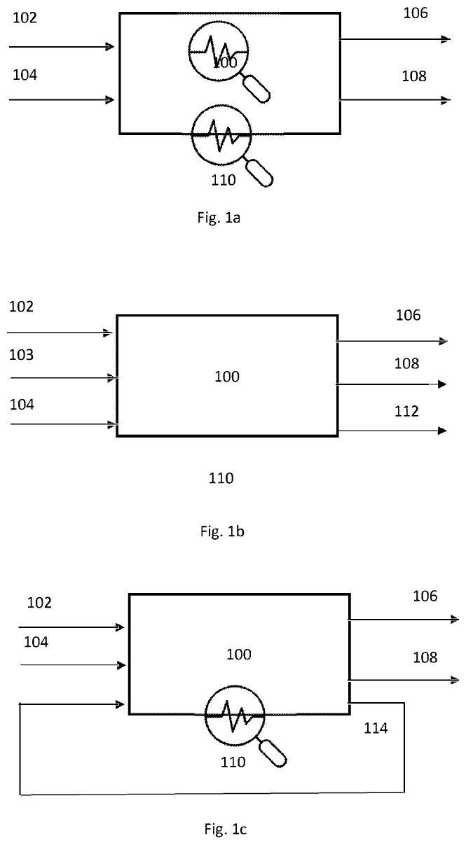

FIGS. 1a-c illustrate examples of chemical processes with multi-input-multi output relations.

Chemical processes 100 may include different process steps for producing one or more output material(s) from one or more input material(s). The chemical process 100 may include at least one process step involving at least one chemical reaction. The chemical process 100 may produce from multiple input materials multiple output materials. Chemical process(es) or process steps include for example oxidation, reduction, hydrogenation, dehydrogenation, hydrolysis, hydration, dehydration, halogenation, nitrification, sulfonation, amination, alkylation, dealkylation, esterification, polymerization, polycondensation, catalysis, fermentation, mixing, separation, purification or the like. The process(es) or process steps may be performed sequentially in time and/or space to chemically, physically, mechanically and/or thermally transform input materials to output materials.

FIG. 1a illustrates input materials 102 and 104 fed to the chemical process 100. The input materials 102 and 104 are chemically processed to output materials 106 and 108. The output materials 106 and 108 may include one main product and at least one by-product. In chemical reactions, the yield of one output material is typically below 100%, because of side reactions and losses upon purification. Hence chemical processes may produce multiple output materials. The main product may signify the product of interest and the by-product may signify the further output product that is unavoidably obtained by the chemical process. The by-product may be an intermediate which can be used as reagent in another chemical process. The chemical process including the feed of input materials and the produced quantity of output materials may be monitored by sensors 110 providing production monitoring data.

FIG. 1b illustrates input materials 102, 103, 104 fed to the chemical process 100. The input materials 102, 103, 104 are chemically processed to output materials 106, 108 as described in the context of FIG. 1a. In addition to the output materials 106, 108 a waste stream 112 may be produced by the chemical process. The waste stream may include any output material that cannot be used as reagent in another chemical process.

FIG. 1c illustrates input materials 102, 104 fed to the chemical process 100. The input materials 102, 104 are chemically processed to output materials 106, 108 as described in the context of FIGS. 1a and 1b. In addition to the output materials 106, 108 a refeed stream of input material 114 may be produced and reused by the chemical process 100.

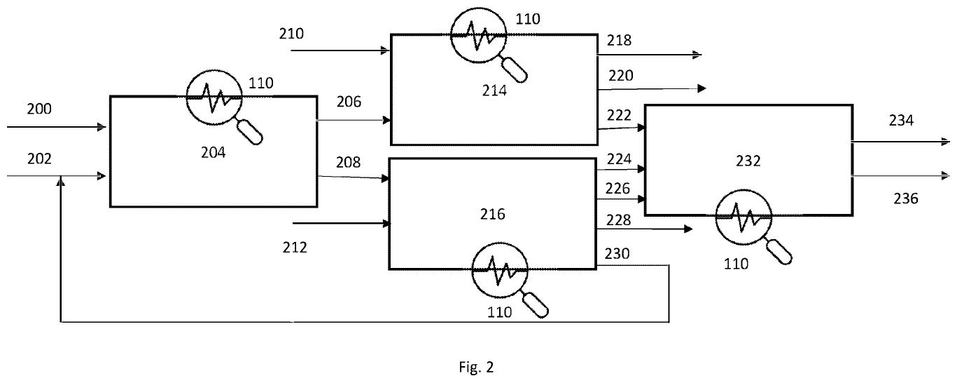

FIG. 2 illustrates a simplified schematic view of multiple chemical processes 204, 214, 216, 232 of the chemical production network.

FIG. 2 illustrates the networked nature of the chemical production network. Multiple chemical processes 204, 214, 216, 232 are interlinked via their input-output material relation. For example, the output materials 206, 208 of chemical process 204 may be the input material of chemical processes 214, 216. Chemical process 214 may produce from the input materials 210 and 206 the output materials 218, 222 and waste stream 220. Output material 218 may exit the chemical production network as end products. The input material 210 may be fed to the chemical process 214 from the outside of the chemical production network. The input material 206 may be fed to the chemical process 214 from the chemical process 204 of the chemical production network. Similarly chemical process 216 may produce from the input materials 208 and 212 the output materials 224-230. Output material 228 may exit the chemical production network as end product. The output material 230 may be recirculated to chemical process 204 and fed into chemical process 204 as input material 202. Chemical process 232 may produce from the input materials 222, 224, 226 the output materials 234, 236. Output materials 234, 236 may exit the chemical production network as end products. This way the chemical production network may use interlinked or interrelated chemical processes to produce output products or end products leaving the chemical production network. The interlinking or interrelation may include at least one intermediate of one chemical process being used as input material to one or more chemical process(es) downstream the one chemical process producing the at least one intermediate.

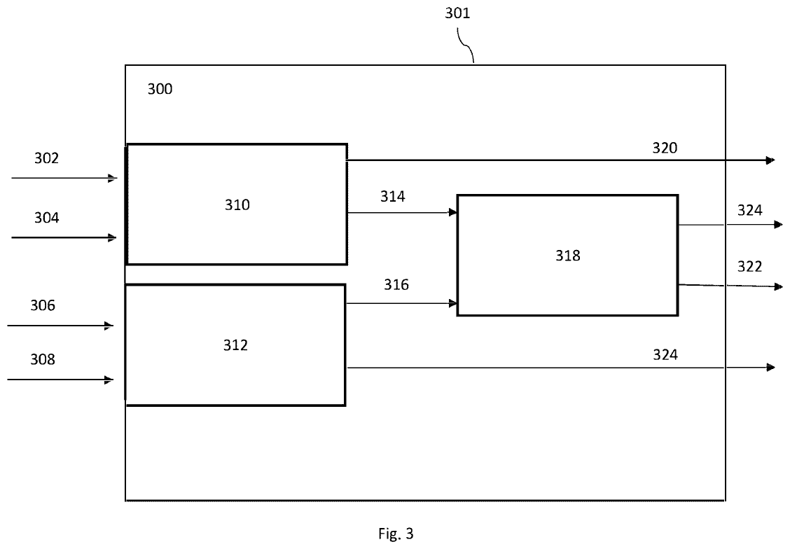

FIG. 3 illustrates a simplified schematic view of a sub-cluster 300 of the chemical production network including multiple chemical processes 312, 310, 318.

The chemical production network may include multiple plants performing chemical processes 312, 310, 318 and forming sub-cluster 300 of the chemical production network. The sub-cluster 300 may be defined by the cluster boundary 301. The sub-cluster boundary 301 may signify input materials entering the sub-cluster 300 and output materials exiting the sub-cluster 300. The input material 302, 304 may be fed into chemical process 310. The input materials 306, 308 may be fed into chemical process 312. The output materials 320, 324 may be provided as end products of the subcluster 300 and exit the subcluster 300. The output materials 314 and 316 of chemical processes 310, 312 may be provided as input materials to chemical process 318. The output materials 322, 324 may be provided as end products of the subcluster 300 and exit the subcluster 300.

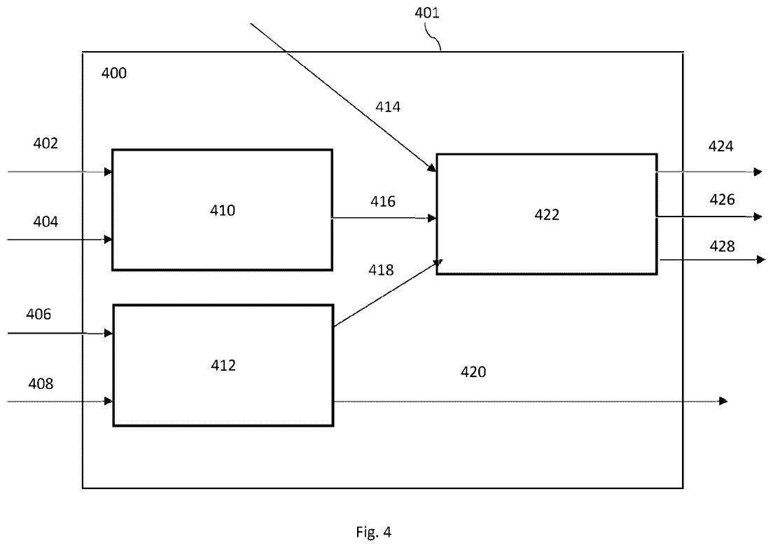

FIG. 4 Illustrates a simplified schematic view multiple sub-clusters 410, 412, 422 forming the chemical production network 400.

The chemical production network 400 may include multiple subclusters 410, 412, 422. The input materials 402, 404, 406, 408 may be fed to subclusters 410, 412. The chemical production network may be defined by the system boundary 401. The system boundary 401 may signify input materials entering the chemical production network 400 and output materials exiting the chemical production network 400. The output material 416 from subcluster 416 and the output material 418 from subcluster 412 may be fed as input material to subcluster 422. In addition, input material 414 may enter the chemical production network 400 and be fed to subcluster 422. The output materials 424, 426, 428 of sub-cluster 422 may exit the chemical production network as end products.

As illustrated in FIGS. 1 to 4 the chemical production network 400 may include multiple chemical processes 100, which may be arranged in subclusters 410, 412, 422. The chemical processes 100 or subclusters 410, 412, 422 may be connected to form a network 400 with multiple production chains interrelated via their material flow. The chemical production network 400 may form part of a discrete product supply chain, wherein the discrete product is produced from one or more chemical outputs, chemical end-products or chemical output materials provided by the chemical production network 400.

The chemical production network 400 may include different entities such as chemical processes 100, chemical plants, sub-clusters 300, or combinations thereof. The chemical production network 400 may include multiple chemical plants including multiple chemical processes. The chemical plant may be operated by an operator associated with the output materials produced by the chemical plant. The sub-cluster of the chemical production network may include one or more, e.g. multiple, chemical plants. The sub-cluster may be operated by an operating entity associated with the input and output materials of the sub-cluster 300.

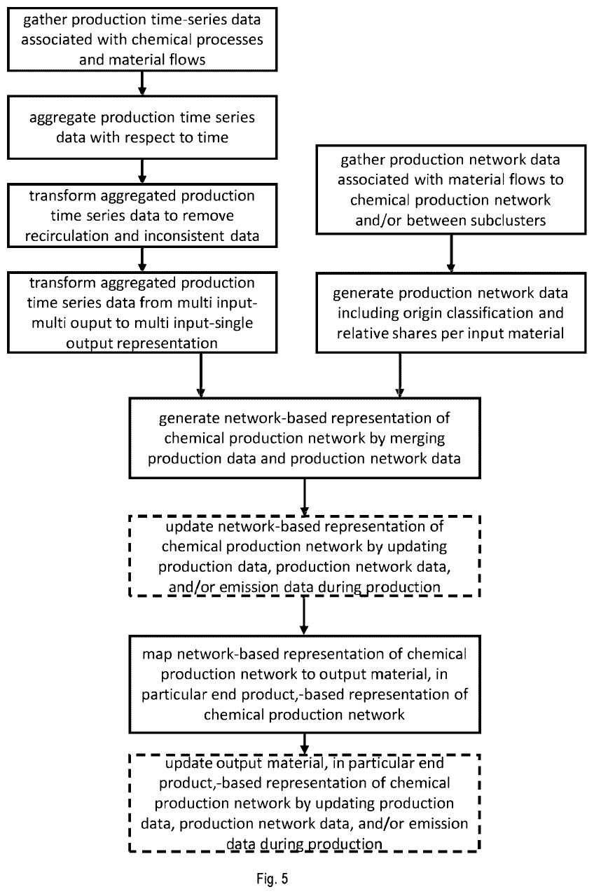

FIG. 5 illustrates an example flow chart of a method for generating digital representations of the chemical production network 400 and/or updating the digital representation for monitoring the chemical production network 400.

For generating the network-based and/or output material, e.g. end-product or product, based representation of a chemical production network different data sets related to different entities of the chemical production network may be gathered and processed. The different entities of the chemical production network 400 may include chemical processes 100, chemical plants and/or subclusters 300 forming the chemical production network 400 as illustrated in FIGS. 1-4.

The gathering of data sets relating to different entities of the chemical production network may include gathering of production data associated with the chemical processes 100 and material flows from input materials to output materials per chemical process 100. The production data may be gathered per chemical process of the chemical production network 400. The production data may relate to production data measured per chemical process 100. The production data may include time series data measured during or on production. The production data may include time series data relating to measured production data according to production recipes per chemical process 100 or chemical process recipes. Production recipes may include measured input quantities per input material and/or measured output quantities per output material of the chemical process 100. The measured production recipes may be aggregated, such as averaged, over time such as per day, week, month, year or multiple days, weeks, months, years e.g. 1, 2, or 3 years. The measured production recipes may be aggregated over time per input material and/or per output material of the chemical process 100. The aggregated production data may be provided per chemical process.

The gathered or aggregated production data may relate to production quantities, such as volumes or amounts, of input material(s) and/or output material(s) per chemical process 100. The aggregated production data may include multi input-multi output relations for one or more chemical processes 100. In some embodiments the time series aggregation of production data is performed prior to generating the digital twin of the chemical production network.

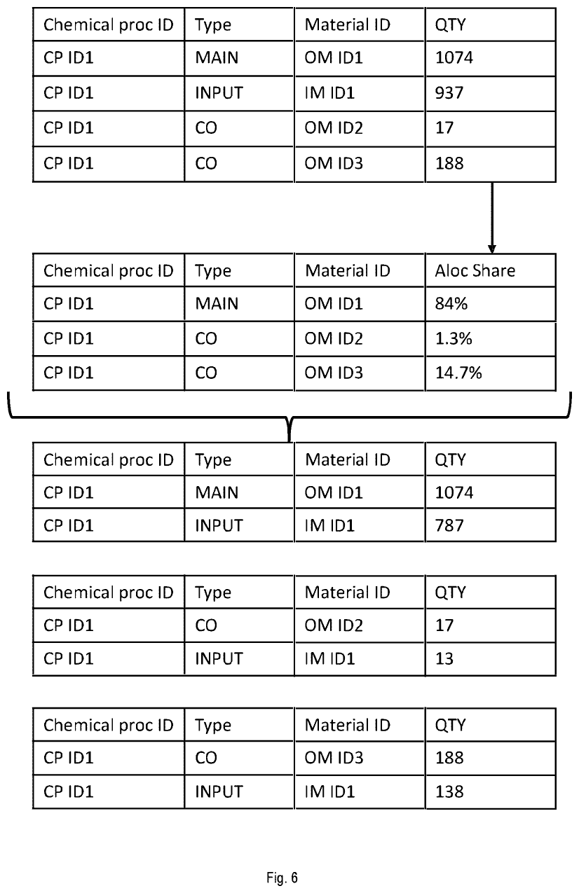

The production data may include chemical process recipes or production recipes associated with the input and output materials per chemical process 100. An example chemical process recipe is illustrated in FIG. 6. The chemical process recipe or production recipe may include process identifiers CHEMICAL PROC ID associated with different chemical processes, input material identifiers IM ID associated with the different input materials, output identifiers OM ID associated with the different output materials, material types TYPE associated with input materials and/or output materials, quantities QTY, such amounts or volumes, related to the input and/or output materials, ratios RATIO (not shown) relating to relative quantities of the input and/or output materials or combinations thereof. The production data may include one or more, preferably multiple, structured data set(s) for chemical process recipe(s) or production recipe(s).

Since chemical processes 100 often adhere to multi-input-multi-output relations the production data provides a representation of a complex multi-input-multi-output network with multiple dependencies between multiple chemical processes 100. Such dependencies may include recirculation within a chemical process, recirculation between chemical processes, dependencies on input materials and output materials between multiple chemical processes and/or splits or forks of output materials to input materials for multiple chemical processes. When merging the production data according to the complex multi-input-multi-output setup of chemical processes 100, the representation of the chemical production network may be difficult to process further and to derive insights on different process setups.

To simplify further processing of the production data, cycles associated with recirculation within chemical processes 100 or in other words recirculation in one chemical process 100 may be removed from the production data. Such cycles may be identified, if one chemical process identifier CHEMICAL PROC ID is related to the same input and output material in the example shown as OM ID and IM ID. Such relation may be identified, if the material type TYPE includes output and input for the same material IM ID, OM ID. IM may denote input material. OM may denote output material. To remove the cycle from the chemical process recipe, the quantities associated with respective material IM ID, OM ID may be transformed to a net quantity related to the material ID with the larger quantity QTY. Further consistency checks may be performed on the chemical process recipes. For example, if one material ID is related to two material types TYPE, the related quantities for such one material ID may be added or subtracted and assigned one material TYPE. Further for example, if the chemical process recipe is inconsistent by not including at least one material type TYPE input or output or by including negative quantities QTY or ratios RATIO, the chemical process recipe may be disregarded and deleted from the production data.

To further simplify processing of the production data, the multi-input-multi-output relations may be transformed to multi input-single output relations by using allocation rules that separate the output materials by mapping the required input material quantities to respective output material. The allocation rule(s) may be determined per chemical process 100 associated with the multi-input-multi-output relation, such as including multiple output material identifiers OM IDs of different type such as MAIN for main output material, BY for by-products, SIDE for side-product or CO for co-products. Chemical processes associated with multi output relations may be identified in the production data by identifying chemical processes 100 associated with multiple output materials. The chemical processes 100 with multiple output materials may be identified by detecting chemical process identifiers associated with at least one main output material and one or more side output material(s). Side output material may be a chemical by-product, which may be marketable or waste. FIG. 6 illustrates an example chemical process recipe, and an allocation rule as may be applied to the example chemical process recipe. Side output material may be specified for instance by the material type TYPE as for example shown in FIG. 6.

In other words, for identification of chemical processes associated with multi-input-multi-output relations the production data may be screened for chemical process identifiers CHEMICAL PROC ID, that relate to multiple material, e.g. input or output material, identifiers. The material identifiers may relate to different types signifying the type of the material identifier as main output, side output, waste or input. If more than one material identifier for one chemical process CHEMICAL PROC ID relate to more than one output type, the chemical process ID may be flagged multi output and may be further processed.

Per chemical process CHEMICAL PROC ID flagged multi-output, one or more allocation rule(s) may be applied. The allocation rule may specify how the input materials related to the chemical process CHEMICAL PROC ID are to be allocated per output material. The allocation rules may be provided by way of a decision tree to select the appropriate allocation for the chemical process. The allocation rules may include process or material specific allocation rules, exceptional allocation rules, volume allocation rules, mass allocation rules, emission allocation rules, stoichiometric allocation rules or combinations thereof. The process or material specific allocation may depend on the specific output material, the specific input material, and/or the specific chemical process. Process or material specific allocation rules may be derived from measurement data related to the specific output material, the specific input material and/or the specific chemical process. Scientifically accepted allocation rules may be provided for the specific output material, the specific input material and/or the specific chemical process. The exceptional allocation rules may include allocation rules with respect to by-products such as waste or residues. The volume allocation rules, mass allocation rules, or stoichiometric allocation rules may depend on the chemical process, in particular the chemical reaction(s) of the chemical process. Emission allocation rules may depend on emission characteristics such as carbon emissions, carbon reuse, carbon capture, waste, energy or combinations thereof. The decision tree for allocation rules may include chemical process CHEMICAL PROC IDs related to material specific allocation rules, exceptional allocation rules, volume allocation rules, mass allocation rules, emission allocation rules or stoichiometric allocation rules. The allocation rule may be selected based on the chemical process CHEMICAL PROC ID flagged multi-output.

On application of the allocation rule, the chemical process recipe may be split per output material OM ID. The input material IM IDs related to the chemical process ID may be allocated per output material OM ID according to the allocation rule. This way, the multi input-multi output relations may be transformed to multi input-single output relations. The transformed production data may thus include a transformed representation of the chemical production network with transformed chemical processes adhering to multi input-single output relations. The thus transformed representation or production data may allow for more simplified and consistent handling and further processing of production data.

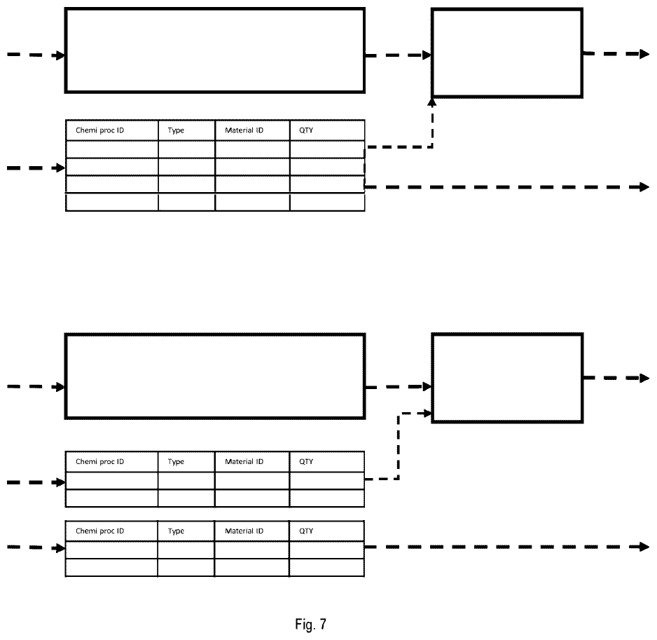

FIG. 7 illustrates an example of connected chemical process recipes as provided by the production data and an example transformation for single output recipes. FIG. 7 illustrates an allocation rule as it may be applied to the chemical process recipe of FIG. 6 to further process multi-input-single-output relations per chemical process depending on the input of the following chemical processes. FIG. 7 schematically shows the structure of interconnected recipes according to the physical process setup.

The upper part of the FIG. 7 illustrates a situation, where one output material related to one first chemical process is used in part as input material for at least one following second chemical process. For example, material identifiers that relate to output material identifier(s) in the first chemical process and corresponding material identifiers that relate to input material identifier(s) in the second chemical process may be identified. This way the use of output materials from the first chemical process in one or more second chemical processes can be detected in the production data to simplify further processing. For example, material identifiers that relate to output material identifier(s) associated with the first chemical process and corresponding material identifiers that relate to input material identifier(s) associated with the second chemical process may be identified. The quantities of output material identifier(s) associated with the first chemical process and corresponding material identifiers that relate to input material identifier(s) associated with at least one second chemical process may be identified. If only part of the quantities of output material identifier(s) associated with the first chemical process are used in the second chemical process, the output material identifier(s) of the chemical process recipe of the first chemical process may be split according to their further use. The chemical process recipe associated with the first chemical process may be split to attribute the quantity of output material in part allocated to the second chemical process as input material. The lower part of FIG. 7 illustrates such split of output materials of the first process according to their use in one or more following chemical process(es). The split may be allocated by quantity, such as mass or volume, used in one or more following chemical process(es). The split thus transforms the multi-input-single output relation into two multi-input-single-output relations per subsequent chemical process. The split thus generates at least two recipes according to the subsequent material flows to one or more chemical processes and/or end products.

Turning back to FIG. 5, the gathering of data sets relating to different entities of the chemical production network may include gathering of production network data associated with material flows to the chemical production network and/or between subclusters of the chemical production network. The production network data may be gathered per subcluster of the chemical production network. The production network data may be generated by gathering material entry data associated with different materials entering the chemical production network such as raw material. The production network data may include inter subcluster data associated with materials transferred between at least two subclusters. The material entry data and/or the inter subcluster data may include time series data relating to material flows into the chemical production network or between subclusters, respectively. The material entry data and/or the inter subcluster data may be time dependent measurement data associated with material flows into the chemical production network or between subclusters, respectively. The material entry data and the inter subcluster data may be aggregated, such as averaged, over time such as per day, week, month, year or multiple days, weeks, months, years e.g. 1, 2, or 3 years. The material entry data may include source IDs, subcluster IDs, chemical process IDs, material IDs and/or quantities per subcluster. The inter subcluster data may include origin subcluster IDs, destination subcluster IDs, chemical process IDs, material IDs and/or quantities per subcluster.

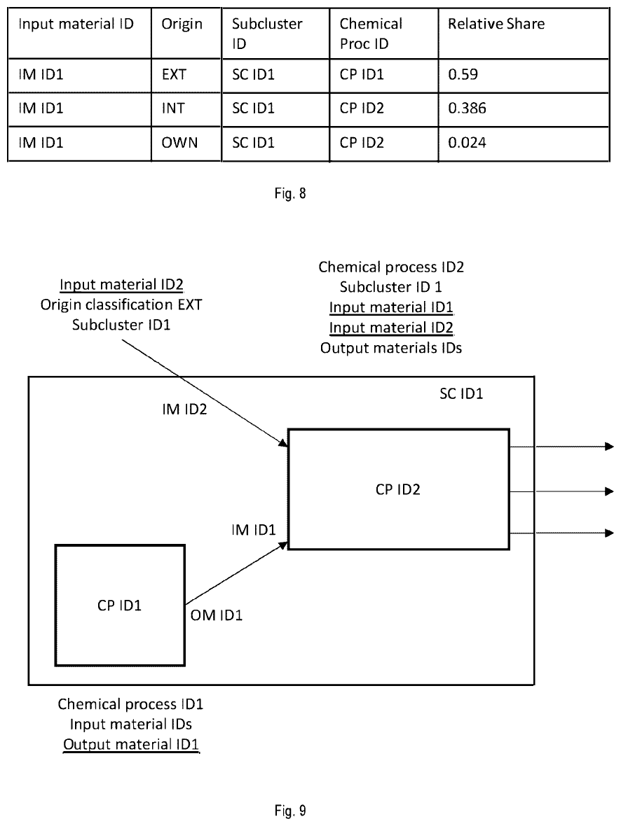

In addition, subcluster consumption data associated with the material consumption per subcluster may be gathered. The subcluster consumption data may include subcluster IDs, chemical process IDs, material IDs and/or total quantities per subcluster. From the material entry data, the inter subcluster data and/or the subcluster consumption, the input material flows per subcluster may be determined. The input material flow may be associated with input material flows to the subcluster. The input material flows may include material flows entering the chemical production network at the subcluster and/or material flows from one subcluster of the chemical production network to another subcluster of the chemical production network. In other words, the material entry data, the inter subcluster data and/or the sub-cluster consumption data may be merged to production network data by determining per subcluster from the material entry data, the inter subcluster data and/or the subcluster consumption data per material identifier ID, an origin classification such as own production, subcluster production, or external, and/or based on the subcluster consumption data the relative share per origin classification. The generated production network data may include material identifier IM ID, origin classification ORIGIN, subcluster identifier SC ID, chemical process identifier CP ID and/or relative share RELATIVE SHARE. FIG. 8 illustrates an example of production network data including origin classification and production network ratios. The generated production network data may be provided for further processing.

FIG. 9 illustrates an example of a network-based representation of the chemical production network.

FIG. 9 illustrates an example of production data and production network data associated with a second chemical process CP ID2 in a first sub-cluster SC ID1. In the example, second chemical process CP ID2 is provided with two different input materials IM ID1 and IM ID2. The first input material IM ID1 is provided within the first subcluster SC ID1 from the first chemical process CP ID1. The second input material IM ID2 is provided from an external source to the chemical production network and specifically to the second chemical process CP ID2. Based on the production data per chemical process and the production network data per subcluster the input material identifiers of the second chemical process CP ID2 can be matched with the material identifiers, e.g. output material identifier OM ID1, of the first chemical process CP ID1 from production data and, e.g. the second input material identifier IM ID2, from production network data. This way the production data and the production network data can be merged to form the network-based representation of the chemical production network.

The network-based representation, as for instance generated based on production data and production network data, may be transformed to a graph data structure. Graph data structures may include vertices (or nodes) and edges connecting the vertices. The vertices may relate to chemical processes and/or sub-clusters. The edges may relate to material flows. The vertices and/or edges may be associated with metadata specifying properties of the vertices and/or edges. The edges may specify a relationship between vertex pairs and optionally an orientation. The graph data structure may be a directed graph structure including orientation of the edges.

The network-based representation of the chemical production network may hence be transformed from an identifier-based data structure (ID structured data) to a graph-based data structure based on material flows (graph structured data). The graph-based data structure may relate to the material flows to or from the chemical processes or subclusters, respectively. The graph-based data structure may relate to input material and output material flows to the chemical processes and/or subclusters, respectively. The graph-based data structure may be a directed graph-based data structure relating the material flows to or from the chemical processes and/or subclusters, respectively. The graph-based data structure(s) associated with each vertex and each edge may reassemble the physical set up of the chemical production network. The graph-based data structure(s) associated with each vertex and each edge may be provided by the production data and the production network data.

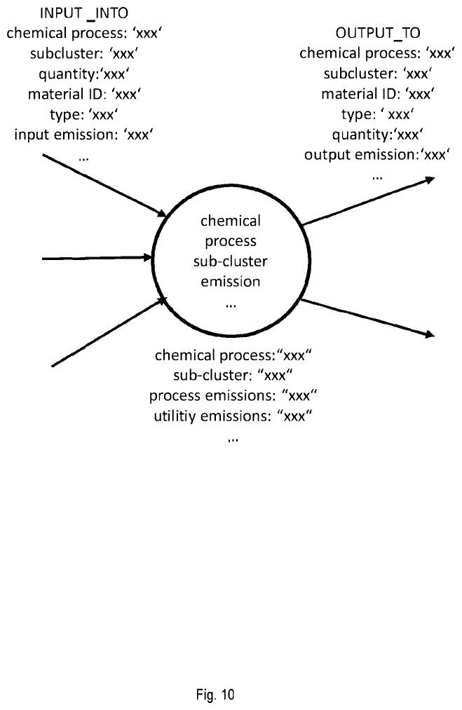

FIG. 10 illustrates an example of a network-based representation of the chemical production network in a graph data structure. FIG. 10 illustrates an example of a graph data structure based on material flows of the chemical production network as provided by production data and production network data. The graph data structure may comprise vertices and edges. Example data structures associated with each vertex and each edge are illustrated in FIG. 10. The vertices may relate to the chemical process. The edges may relate to the input or output material flows to or from the chemical process. Other graph-based data structures are possible. For example, the vertices may relate to the sub-cluster including chemical processes associated with the subcluster. The edges may relate to the input or output material flows to or from the subcluster. By using the graph-based data structure the level of detail may be dynamically adjusted by combining vertices and edges associated with chemical processes of one subcluster to a subcluster-based vertex and edge. By using the graph-based data structure the level of detail may be dynamically adjusted by expanding vertex and edge associated with one subcluster to multiple vertices and edges associated with the chemical processes of the one subcluster. Such graph-based data structure may be used for visualization of the chemical production network by way of vertices and edges associated with metadata or a selection of metadata.

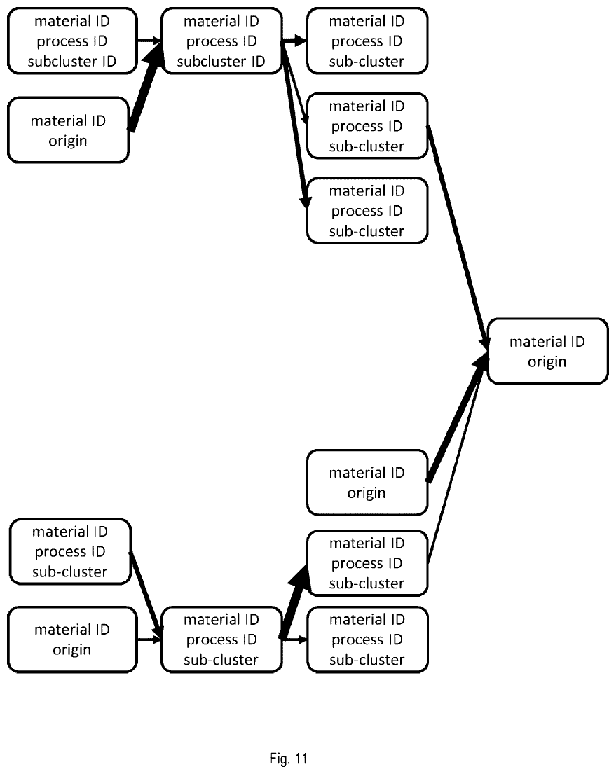

FIG. 11 illustrates an example of a network-based graph representation of the chemical production network as may be displayed on a user interface. FIG. 11 illustrates an example of a monitoring material flows of the chemical production network as provided by production data and production network data. Material flows may in this case relate to input material quantities as provided to the chemical production network, the sub-cluster(s) and/or the chemical processes. The material flows may relate to subclusters and/or chemical processes in combination with their respective input or output material flows. For example, in FIG. 11 the nodes may signify subclusters. The triplet data may signify material flows between subclusters. The duplets data may signify material flows from external suppliers to subclusters. The arrows may signify the quantity of respective material flows between subclusters and/or from external sup-pliers to subclusters. The visualization illustrates how the network-based representation of the chemical production network allows for monitoring internal and external materials flows of the chemical production network.

To monitor the material flows of the chemical production network, production network data may be gathered, e.g. in real time or over a time period, from entry points to the chemical production network and/or from entry points to sub-clusters. On entry of input materials to the chemical production network and/or to subclusters, the quantity, such as mass or volume, may be measured. Such measurement may be provided per input material identifier per subcluster and/or per input material identifier per raw material provided to the chemical production network to generate production network data. The production network data may be generated by classifying the measured quantity per input material identifier, per subcluster and/or per raw material. The data structure merging production data and production network data or the network-based representation of the chemical production network may be updated based on input material as provided to the chemical production network or per subcluster. The data structure merging production data and production network data or the network-based representation of the chemical production network may be updated on providing input material to the subcluster or the chemical production network. The data structure merging production data and production network data or the network-based representation of the chemical production network may be updated, e.g. in real-time or over a time period, per subcluster e.g. for material provided internally to the sub-cluster from a different sub-cluster or externally to the chemical production network.

For more detailed monitoring of the material flows of the chemical production network, production data may be gathered, e.g. in real time or over a time period, from chemical processes. For example, the input materials processed per chemical process such as the quantity, e.g. mass or volume, of input material processed per chemical process may be measured. Such measurement may be provided per input material identifier and per chemical process to up-date production data per chemical process. The data structure merging production data and production network data or the network-based representation of the chemical production network may be updated based on input material as provided per subcluster and/or chemical process. The data structure merging production data and production network data or the network-based representation of the chemical production network may be updated on providing input material to the subcluster and/or chemical process. The data structure merging production data and production network data or the network-based representation of the chemical production network may be updated, e.g. in real-time or over a time period, per subcluster and/or chemical process. The data structure merging production data and production network data or the network-based representation of the chemical production network may be updated per subcluster and/or chemical process during production.

The monitoring of the material flows of the chemical production network may further be adapted to monitor resource usage, such as environmental impact, of the chemical production network. This may be done based on the updated material flows as described above. The resource usage data may include or relate to material flows, such as material quantities, e.g. input material quantities. The resource usage data may be updated with respect to the chemical processes and/or the input materials. The resource usage data may relate to environmental impact of the chemical production network. The resource usage data may include emission data, such as external emission data related to input materials, energy data related to the chemical processes or direct emission data related to the chemical processes may be measured to update resource usage data. More details on adapting or using the network-based representation of the chemical production network to monitor the resource usage, such as the material flows and/or the environmental impact of the chemical production network will be lined out in more detail below.

Turning back to FIG. 5, the network-based representation that maps the chemical production network according to material input-output relations of the chemical production network may be generated. The material input-output relations may be based on the production data associated chemical processes and the production network data associated with subclusters. The materials input-output relations may include input-output relations per chemical process including the origin of the input material. The origin classification may specify the origin of the input materials provided to the subcluster. The input material origin or origin classification may relate to input material provided to the chemical production network (external), input material provided inside the subcluster (internal), or input material provided to the subcluster by another subcluster of the chemical production network (inter subclustered). The material input-output relations may include a mapping of input materials to output materials including the origin classification for input materials and the relative share per origin classification. The production data may include input-output relations per chemical process. The production data may include a subcluster ID per chemical process. The production network data may include subcluster IDs, chemical processes per subcluster, input materials per subcluster, the origin classification of input materials and/or the relative share per origin classification. The material input-output relations of the chemical production network may be determined by combining the production data and the production network data based on the input materials per subcluster and per chemical process. In other words, the production network data adds a further dimension to the production data by providing the origin classification of respective input materials and the relative share per origin classification. Hence the production data is further enriched by adding on subcluster level the material origin or origin classification of input materials and the relative share per material origin or origin classification.

The generated network-based representation may represent the chemical production network by subcluster, chemical process, input material, input material origin, output material and/or at least one interrelation between input material and output material. The interrelation may be provided by quantity or ratios per input material and/or per output material per subcluster and/or per chemical process. The network-based representation may be transformed to a directed graph data structure including subclusters and/or chemical processes as vertices and material flows as edges. The edges may be directed according to the input material flow to the subcluster and/or chemical process or according to the output material flow from the subcluster and/or chemical process. The vertices and/or edges may be associated with meta data signifying the subcluster and/or chemical processes per vertex and the input material flow to the subcluster and/or chemical process or the output material flow from the subcluster and/or chemical process per edge. The data structure merging production data and production network data or the network-based representation of the chemical production network may be updated per subcluster and/or chemical process during production as e.g. described in the context of FIG. 11.

As illustrated in FIG. 5, the network-based representation may be transformed to an product or output material-based, e.g. end-product-based, representation that maps the production network according to output material relations. The output material relations may relate to end-product relations. The output material relations may be associated with the resource usage required to produce a certain output material, e.g. end product. The resource usage may include input material usage or process usage. The input material usage may relate to the quantities of input materials provided to the chemical processes and/or subclusters. The process usage may relate to utilities, such as energy or steam, or emissions related to utilities, such as onstage greenhouse emissions of the chemical processes. The output material, e.g. end-product, relations may be determined from the input-output relations providing the resource usage, e.g. associated with input materials and/or chemical processes, for producing the output materials, e.g. end products, of the chemical production network. The network-based representation may be transformed from a network flow logic to a material use logic. The network flow logic may relate to material flows from input materials to output materials per chemical process or material flows according to the physical setup of the chemical production network. The material use logic may relate to the output materials, e.g. end products, produced by the chemical production network from input materials per chemical process involved in the production of the output material, e.g. end product.

In other words, the network-based representation may adhere to the network logic by providing input material flows per chemical process and/or per sub-cluster. In simple words, the network logic may adhere to the quantity of output material, e.g. end product, produced for a given quantity of input materials per chemical process involved in the production of the end product. The output material, e.g. end-product, based representation may adhere to the output material, e.g. end-product, logic by providing total production quantities, e.g. input material quantities, per output material, e.g. end-product. In simple words, the output material or end-product logic may adhere to the quantities of input material(s) needed to produce a given quantity of output material or end product. The output materials or end-product based representation is advantageous, since the output material or end-product logic gives transparency into resource usage contribution per output material or end product rather than resource usage contribution per input material of the chemical production network. The output material or end-product based representation, hence, provides a consolidated input factor per output material or end-product rather than a network-based factor per input.

This way the resource usage associated with input materials and/or chemical processes can be made transparent and monitored increasing the ability to monitor resource usage, in particular the material usage and/or environmental impact, per output materials, e.g. end-product. The product or output material, e.g. end product, based representation of the chemical production network may be adapted to map environmental contributions of the chemical processes, including respective input and intermediate materials, involved in the production of the output material or end-product to an output material or end-product related environmental property. For monitoring the environmental impact, the representation may be adapted for allocating environmental properties involved in the production of the output material, e.g. end product, to the total environmental property of the output material, e.g. end product.

The transformation of the network-based representation to the product or output material, e.g. end product, based representation is for complex chemical networks very time consuming and processing intensive, since a matrix inversion is required. For example, based on an input-output model the equation v=(E−A)*p may be rearranged to p=(E−A)⁻¹*v. In the model p may relate to a total production vector relating to end products, E is the identity matrix, v may relate to a demand vector relating to end products and A may relate to an attribution matrix including input-output factors or relations. Based on the input-output model resource usage e.g. in terms of input materials for the production of end products of the chemical production network may be determined.

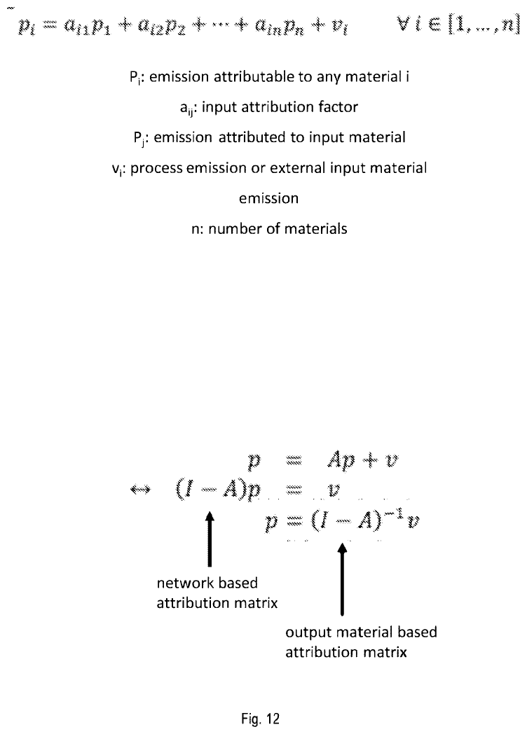

FIG. 12 illustrates an example of an input-output model for generating the network-based representation.

The input-output model may be translated to monitor environmental impact of the chemical production network. FIG. 12 illustrates one example of the input-output model for the example of emission contributions associated with the chemical production network to monitor resource usage in terms of emission. Environmental contributions may relate to greenhouse gas emissions or carbon emissions reflected in carbon equivalents (Co2 eq). According to the Green-house Gas Protocol Standard or the European Commission Product Environmental Footprint (PEF 2021) three scopes are defined: Scope 1 may relate to Co2-eq emissions from chemical production within the system boundary of the chemical production network. For example, scope 1 emissions may include emissions from chemical processes, incineration and/or waste treatment at plant or sub-cluster level of the chemical production network. Scope 2 CO2-eq emissions may relate to the generation of purchased energy, such as electricity and/or steam used to power plants and/or chemical processes of the chemical production network. Scope 3 CO2-eq emissions may relate to input materials or other resources provided to the chemical production network.

Carbon footprints may be calculated according to international standards such as ISO 14064-1:2019, ISO 14064-2:2019, ISO 14064-3:2019, ISO 14067:2019, ISO 14040:2006, ISO 14044:2006, ISO 14040:2006/AMD 1:2020, ISO 14044:2006/AMD 2:2020 for Life cycle assessment or ISO 14067:2018 for Product Carbon footprints (PCF); or also sectoral standards such as Together for Sustainability's “PCF Guideline for the chemical industry” or the Catena-X PCF Rule book; or according to “Pathfinder Framework: Guidance for the Accounting and Exchange of Product Life Cycle Emissions” issued by the Partnership for Carbon Transparency powered by WBCSD. The basic equations for such calculations are e.g.
for input material kg Co2-eq=Amount of activity×Emission factor×Global warming potential with “activity data” being a quantitative measure of a level of activity that results in GHG emissions such as kg input material used and “emission factor” being a factor that converts activity data into GHG emissions, or
for chemical processes kg Co2 eq.=Direct emission×Global warming potential with global warming potential being provided by a data base configured to store and provide global warming factors.

The activity factors may relate to material, e.g. input material, transport and/or energy associated with the chemical production network. Direct emission factors may be determined at least in part from monitoring data or production data associated with the chemical production network. Carbon removals or avoidance through the use of biogenic carbon, land use, carbon capture storage, carbon capture and carbon utilization or any activity relating to sequestration or absorption of GHG emissions may be considered as a negative Co2 eq.

Since the environmental contributions as measure of environmental impact depend on multiple factors, emission data may be gathered from different parts of the chemical production network. Emission data may include multiple data sets related to different emission contributions associated with Scope 1, 2 and 3 emissions. Emission data may be gathered from distributed monitoring systems of the chemical production network. Emission data may relate to emissions associated with the production of output materials by the chemical production network. Emission data may be associated with emissions related to chemical processes, input materials fed to chemical production network and/or energy used for production.

Input emission data may be gathered from a data base related to the input material provider(s) (e.g. Scope 3). Input emission data may relate to the input materials used by the chemical production network to produce output materials. Input emission data may relate to the type of input material, a quantity of input material, such as mass, volume or amount, per type, a time period for the quantity of input material, such as aggregation period of e.g. hours, weeks, days, months or years, a location related to the use of the input material and/or a location related to the manufacture of the input material, technological specification per input material type such as concentration. Input emission data may in addition relate to non-production related inputs provided to operate the chemical production network such as use of computing resources. Input emission data may relate to emission factors related to input material types. Input emission data may include the product carbon footprints (or Co2 eq. for input material type) associated with the input materials entering the chemical production network and/or used for chemical processes. Input emission data may include the total carbon emissions (or Co2 eq. for supplier) associated with the input materials entering the chemical production network and/or used for chemical processes.

Direct emission data may be gathered from plants and/or chemical processes of the chemical production network. Direct emission data may be associated with the carbon emissions or greenhouse gas emissions generated by the operation of the chemical production network. Direct emission data may be associated with the chemical processes and the carbon emissions or greenhouse gas emissions generated by operating the chemical process (main process emissions).

Transport emission data may be gathered from subclusters and/or plants of the chemical production network (e.g. Scope 1 or 3). Transport emission data may be derived from production data and/or production network data relating to material flows of the chemical production network (e.g. Scope 1 or 3). Transport emission data may be derived from the merged production data and production network data. For example, the material flows between subclusters or to the chemical production network may be used in connection with the location of the subcluster or the location of a supplier to determine transport emissions.

Output emission data may be gathered from chemical processes, plants and/or subclusters. Output emission data may include any output material produced by the chemical processes, plants and/or subclusters that exits the chemical production network and is disposed or treated. For example, waste or wastewater emission data may be gathered from a data base related to the chemical processes, plants and/or subclusters (e.g. Scope 1). For example, waste or wastewater emission data may be associated with the chemical production network and may include measured or provided data related to waste or wastewater generated by the chemical production network (e.g. Scope 1).

Energy emission data may be gathered from chemical processes, plants and/or subclusters. Energy emission data may relate to any energy consumption of brown or green energy e.g. from wind farms. For example, energy data may be gathered from a data base related to the input energy provider (e.g. Scope 2). Energy emission data may relate to any energy consumption of energy produced inside the chemical production network or as part of the chemical production network. For example, energy data may be gathered from a data base related to the energy genera-tion of the chemical production network for the production of materials (e.g. Scope 1).

The emission data may be gathered from different chemical processes, plants and/or subclusters of the chemical production network. Emissions data sets may be gathered from different chemical processes, plants and/or subclusters of the chemical production network. Emissions data sets may be gathered on different hierarchy levels of the chemical production network. Direct emission data may be gathered per chemical process, plant and/or sub-cluster. Transport emission data may be gathered per chemical process, plant and/or sub-cluster. Input emission data may be gathered per chemical process, plant and/or sub-cluster. Output emission data may be gathered per chemical process, plant and/or subcluster. Energy data may be gathered per chemical process, plant and/or sub-cluster.

The emission data collected e.g. for different hierarchy levels of the chemical production network may be mapped to the network-based representation of the chemical production network. Such mapping may include mapping of emission data to chemical process(es), input material(s), plant(s) and/or subcluster(s).

In one example, the product carbon footprint of input materials provided to the chemical production network may be provided via a data base associated with the material supplier. The product carbon footprints of the input materials may be allocated to the input materials provided to the chemical production network. The product carbon footprints may be allocated according to the input material quantities provided to the chemical production network data.

In another example, the direct emissions associated with chemical processes may be provided per subcluster and/or plant. The direct emissions may be mapped to chemical processes. For mapping an allocation rule may be used. The direct emissions may be mapped according to a share per chemical process to the subcluster and/or plant. The share may be determined based on the quantity of energy or utility usage of the chemical process and/or plant.

In another example, the transport emissions associated with transport inside the chemical production network may be provided, such as transport between subclusters or inside subclusters. The transport emissions may be deter-mined from the location of subclusters, chemical processes and/or plants as provided by or derivable from production network data. The transport emissions may be determined separately from the input materials/output materials trans-ported. The transport emissions may be mapped to the input materials or output materials transported.

In another example, the energy emissions associated with energy supplied to the chemical production network may be provided, such as energy supplied to or used by subclusters, plants and/or chemical processes. The energy emissions may be determined from the energy supplied to subclusters, chemical processes and/or plants. The energy emissions may be determined based on the origin of the energy. The energy emissions may be mapped to the sub-clusters, plants and/or chemical processes. The energy emissions may be allocated or attributed to the subclusters, plants and/or chemical processes by usage share, production volume share and/or production cost share.

The emission data may be mapped to input material(s), output material(s), chemical process(es), and/or transport(s). This way the production data and production network data may be further enriched with emission data. For example, the input materials entering the chemical production network may be related to emission data associated with such input materials. In particular the input material identifier for the input material type may be linked to emission data for the respective input material type. Further for example, the chemical processes operated by the chemical production network may relate to emission data associated with such chemical processes. In particular, the chemical process identifier for the chemical process type may be linked to emission data for respective direct emissions. Further, the chemical process identifier for the chemical process or chemical process type or the subcluster identifier for the sub-cluster or subcluster type may be linked to emission data for respective energy usage. Further for example, the trans-ports of input/output materials inside the chemical production network may relate to emission data associated with such transports. In particular the input/output material identifier for the input/output material transported inside the chemical production network from one location to another may be linked to emission data for the respective trans-ports.

The emission data may be attributed per chemical process or input material per chemical process as lined out in WO2022073935A1, the disclosure of which is herewith incorporated by reference.

Based on the mapping of emission data, the network-based representation of the chemical production network may be adapted to monitor emissions of or emission contribution(s) to the chemical production network. To interpret the model translated to monitor environmental impact of the chemical production network as shown in FIG. 12 three scenarios may be considered:
1) If i signifies any produced output material, then aij is an input factor regarding the required inputs to produce the output material, pj relates to the carbon footprint of the respective input material (e.g. scope 1 or 3) and vi relates to the process emission of the chemical process step including direct emission (e.g. scope 1) and energy emission (e.g. scope 2).
2) If i signifies any used input material, then aij is a relative share of the consumption of the input material, pj is the carbon footprint of the produced input material (e.g. scope 1) or the externally provided input material (e.g. scope 3) and vi=0.
3) If i signifies any externally provided input material, then pi=vi with vi being the carbon footprint of the externally provided input material.

Hence equation (1) as provided in FIG. 12 may relate the environmental contributions per material, such as input material produced by subcluster, input material produced by different subcluster, input material provided to chemical production network or output material produced by chemical production network. The attribution factors A may relate the emission contribution by usage of input material. The emission vector v may relate to the direct emission of the process producing the output material or the external input material emission.

Based on the gathered emission data, including for example input emission data signifying the emission contribution per externally provided input material, direct emission data signifying the direct emissions per chemical process or sub cluster, energy emission data signifying the emission contribution related to externally provided or internally produced use of energy per chemical process or subclustered and output emission data signifying the output emission contributions not related to materials further used, and the network-based representation of the chemical production network, the different emission contributions may be related according to Equ. 1 to provide the network-based attribution matrix or the product or output material-based attribution matrix as illustrated by Equs. 2.

In other words, the network-based representation may include chemical processes, externally provided input materials per chemical process, internally provided input materials per chemical process and respective quantities of input materials. Similarly, energy and transport contributions may be taken into account. The product carbon footprint of the output material produced may be determined by the product carbon footprint of the input materials and the emissions related to the one or more chemical processes. The product carbon footprint of the input materials may be determined by the quantity of input material used per input material type and the product carbon footprint per quantity of the input material per input material type. The product carbon footprint of the input material used may be determined by the share of the input material provided to the chemical production network and/or the share of the input material provided from one chemical process, plant or sub-cluster of the chemical production network to another following chemical process, plant or sub-cluster of the chemical production network. The product carbon footprint of the input material provided to the chemical production network may be determined by the product carbon footprint of the input material. This way the environmental impact of the chemical production network may be determined based on the network logic of input materials, chemical processes and/or output materials. Such network-based representation can hence provide product carbon footprints for output materials, input materials provided inside the chemical production network and/or input materials provided to the chemical production network. However, the network-based representation adheres to the logic of product carbon footprint contributions to the product carbon footprint per chemical process and input material. The network-based representation is not suitable to provide the product carbon footprint contributions by the quantity of input material and upstream production required to produce the output material that exists the chemical production network.

For example, the emissions vector v related to emissions associated with the chemical process or the input material entering the chemical production network may be mapped to the total emissions vector p related to emissions associated with any input or output material. In particular, the latter provides for transparency and the ability to monitor resource usage and in particular, the environmental impact, per end-product. Thus, the attribution factor matrix (I-A) needs to be inverted. The inverted attribution factor matrix (I-A)⁻¹ provides with each entry the factor which the corresponding v-entry needs to be multiplied. Since v includes the chemical process emissions and the externally purchased input material PCFs, each factor of the inverted attribution matrix (I-A)⁻¹ provides the amount of externally purchased input material or the upstream process needed to produce one kg of the end product. In other embodiments other emission contributions may be reflected by Equs. 1 or 3.

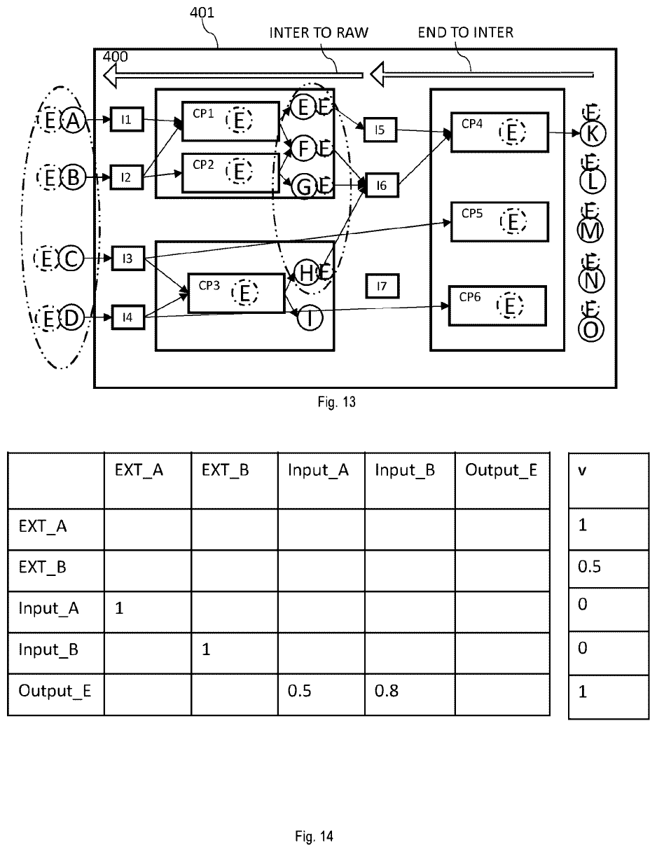

FIG. 13 illustrates an example extract of the network-based representation as generated from fork-free or loop-free chemical process recipes and production network ratios. FIG. 13 illustrates an example of a data structure for relating the environmental contributions to material flows.

For simplification and illustrative purposes FIG. 13 only illustrates some interconnections between chemical processes CP1-6, materials A-O (solid circles) and associated emission contributions E (dashed circles). As described in the context of FIG. 12 the environmental contributions E associated with input materials A-I and chemical processes CP1-6 used to produce the output materials E-O may be provided by solving the matrix equations of FIG. 12. The output materials E-O may be produced by the chemical production network. The output materials E-O may be produced by one or more chemical process(es) CP1-6 and/or one or more input material(s) A-I. In other words, the output materials E-O may be produced within the system boundary of the chemical production network. The output materials K-O may be intermediate materials E-I produced or end products K-O exiting the system boundary of the chemical production network. The chemical production network may be represented by chemical processes CP1-6 converting input materials A-I to output materials E-O. Per chemical process CP1-6 the chemical process recipe may represent the input material(s) A-I and the output material(s) E-O. The input materials A-I may include input material E-I produced by the chemical production network and/or input material A-D provided to the chemical production network. In the latter case the input material A-D may enter the system boundary of the chemical production network. The output material E-O may include the end product K-O, if it leaves or exits the system boundary of the chemical production network. The output material E-O may include intermediate products E-I, which may be used as input material by another or linked chemical process CP1-6.

The chemical processes CP1-6 may be linked via their associated chemical process recipes and production network ratios I1-7 or interconnection ratios or consumption mixes. Production network ratios I1-7 may signify the origin of the respective input material A-I and the quantity ratio I1-7 per origin related to the total quantity of the respective input material A-I provided to the respective chemical process CP1-6.

For generating a production path and/or value chain of the end products K-O, the end product denoted K may be chosen as starting point. The end product K is as an example illustrated by reference sign K signifying the exit of the chemical production network 400. The production path and/or value chain is generated by linking the end product K to input materials A-C and E-H. In other words, the production path and/or value chain is generated by concatenating input materials A-C and E-H of the recipe for the end product K with the output of another recipe according to the production network data I1-3 and I5,6 until an input material A, B, C entering the chemical production network is reached. In the present example the production path from end product to intermediate END TO RAW and from inter-mediate to raw materials INTER TO RAW illustrated in FIG. 13 may for example include:

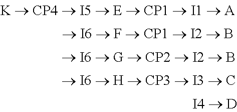

The production path may be generated based on the network-based representation of the chemical production network 400, which may be fork-free and recirculation-free on chemical recipe basis. This way a single output digital production path may be provided via the network-based representation of the chemical production network. The recipes may include lists with input materials indicated by a product identifier and corresponding ratios that are used to produce output material by the chemical process. The recipes may map chemical processes and materials. The production network data may include lists that indicate the different sources of input material and the ratio for the input material from different sources as provided to the chemical processes. The production network data may include lists that indicate the different sources per sub-cluster of the chemical production network.

The production path may be generated from the end product K to the input material A-D entering the chemical production network in an opposite direction to the production direction (END TO INTER, INTER TO RAW). This may also be referred to as downstream direction. The production path may include the input materials A-I and output materials I-O as provided by chemical process recipes per chemical process CP1-6 and interconnection ratios I1-7 signifying the consumption mixes or network ratios related to the input material origin.

The chemical processes CP1-6 and the input materials A-O may be associated with environmental contributions E (dashed circles). In FIG. 13 the numbers in the circles drawn as broken lines indicate the environmental contributions E either per input material A-O and/or chemical process CP1-6. In an example the numbers indicate the raw material PCF E and/or the chemical process emissions E.

In the following a more detailed example is provided, to demonstrate the mapping of the environmental contribution of the input materials A-I and the chemical processes CP1-CP6 to the environmental property of the end-product K-O.

The network-based representation illustrated in FIG. 13 may be the network-based representation of the chemical production network provided together with environmental contributions as lined out above. The network-based representation of the chemical production network may be provided from an external source, such as a memory and/or a storage device and/or from an internal source, such as another device or module of an apparatus for generating a digital twin.

For determination of the environmental property of the end product K, a mathematical model may be used, that con-solidates environmental contributions E along the value chain and/or production path in order to generate the end product K related environmental property E. The value chain may comprise multiple recipes, that may be linked ac-cording to the material flows between chemical processes CP1-6. The recipes may define how input materials, e.g. chemicals, are combined in order to create an output material E-O or the end product K. The value chain may comprise a series of recipes which define by their substantially serial connection how input materials A-I are combined to create output material E-O or the end product K. The value chain may also comprise the production network data I1-7 that may define from where a specific chemical is sourced. As shown in FIG. 7 by eliminating forks and/or loops in a recipe it may be assumed that one single recipe of the chain of recipes may substantially generate a single output material, e.g. a single chemical. The environmental contributions may comprise raw material emissions, process emissions and/or transport emissions.

The raw material A-D may enter the value chain at the system boundary. The raw material A-D may be associated with raw material PCFs E. The chemical processes CP1-6 may convert multiple raw materials A-D to multiple output materials E-O. The chemical processes CP1-6 may be associated with process emissions E, that may arise from the production step of the value chain. Transport emissions may substantially be caused by transport activities of the materials inside the system boundary of the chemical production network and needed along the value chain. Transport emissions are not explicitly shown for simplification purposes and may be considered part of the input material contributions.

The network-based representation of the chemical production network may be used to distribute and/or map the environmental contributions E along the value chain of the end product e.g. K. The network-based representation of the chemical production network may be used for mapping of raw material, process and/or transport emissions E. The network-based representation of the chemical production network may be applied for monitoring and/or determine PCF of end product e.g. K. In the simplest version the production path illustrated in FIG. 13 may be used to accumulated the environmental contributions to the total PCF of product K. However, such contributions are added according to the network logic following the chemical processes and respective material flows. Such representation makes the monitoring of the total environmental property of the end product difficult and requires more intensive analysis to determine the individual contributions specifically per input material.

To simplify monitoring of environmental properties of end products, the network-based representation may be trans-formed to an end-product based representation. In production processes input-output-models can be used to determine the production volumes needed to satisfy a given demand. An example of an input-output-model in general has the format:

$${x}_{i} = {z}_{i 1} + … + {z}_{i n} + {f}_{i} , \qquad ( Equ . 1 )$$

$$i = 1 , … , n$$

where x is the production, z is the consumption and f the external sale. Such model may be provided to maps input material-output material relations by determining the input factor matrix:

$${p}_{i} = {a}_{i 1} {p}_{1} + {a}_{i 2} {p}_{2} + … + {a}_{in} {p}_{n} + {ν}_{i} \qquad ( Equ . 2 )$$

$$∀ i ∈ [ 1 , … , n ]$$

where aᵢⱼ is the input factor or attribution matrix relating to the amount of material i required to produce one unit of material j, pᵢ is the total production of material i and vᵢ is the demand for material i. The logic of the input-output-model can be used for mapping resource contributions such as material resource contributions or environmental contributions. In particular, the input-output model logic may be used for determining the environmental property of the end product and in this way may be used to monitor end product environmental impact in terms of PCF values. For example, retrieved environmental contributions, e.g. carbon emissions, can be interpreted as a cost to be allocate and/or mapped according to the input-output model. To generate a network-based representation of the chemical production network a matrix may be assembled according to Equ. 2 of the input-output-model. For determining the environmental property of end products, the environmental contribution from chemical processes and/or input materials used to produce the end product may be added according to the input-output model.

FIG. 14 illustrates another example extract of the network-based representation as generated from fork-free or loop-free chemical process recipes. FIG. 14 shows a partial view of the network-based representation of the chemical production network assembled on the basis of the input-output-model including the input attribution matrix.

The network-based representation or network-based attribution matrix of the chemical production network may be based on the complex process network of FIG. 1-4 or 13. In matrix form the rows and columns of the network-based representation may correspond to the different materials that appear in the different stages of a value chain and during the production of the end product in the process network. In other words, the columns and rows of the network-based representation may relate to the inputs and outputs of the chemical process recipes and/or the production network data. The network-based representation may include an overall matrix summarizing the production data and/or production network in a structured and/or indexed data structure.

For example, the externally received input material A, referred to by Ext_A, may relate to the input material to the chemical production network and may be provided by production network data. The input material may be associated with the raw material environmental property or PCF value of 1. This value 1 may be entered in the environmental property vector v at a row indicate by the origin, i.e. Ext_A. The externally purchased product A may be used as input material Input_A for chemical process. It may be associated with the origin classification external. Additionally, the ratio of externally purchased input material may be provided by production network data. The production network data may provide the example value of 1. Production network data having the value of 1 may mean that there is no other origin for input material than the external. As there is no share of internal input materials, the total amount is concentrated to one tree. The interconnection ratio value may be entered at the corresponding column and row in the network-based matrix representation, i.e. entered at Ext_A, Input_A. Input material B Ext_B may be related to the environmental contribution of the raw material. This may be signified by the PCF value of 0.5. The environmental contributions of raw material A and B may be entered in the environmental property vector column v, indicating environmental contributions per input material per chemical process.

Ext_A and Ext_B may be transported between two chemical processes as indicated by production network data and used as input Input_A and Input_B in the chemical process CP1 according to the loop free aggregated multi-input-single-output representation. Input_A may contribute with a ratio of 0.5 and Input_B may contribute with a ratio of 0.8 to the output material E, i.e. Output_E. The output material E may be classified as OWN_PRODUCTION as it is generated by chemical process of the chemical production network. Process emissions of a value 1 may relate to the chemical process which may be entered in environmental property vector v, indicating the environmental property of the chemical process.

Three different cases can be differentiated, how process data, e.g. recipe data or production network data, input material and output material, intermediate material or end products and their possible emission contributions may be added to the network-based representation.

If the recipe is to be added to the matrix 1500 then the output product K, Ext_A, Ext_B and/or produced material of the recipe is added to the row i, e.g. Output_E. The input for this specific recipe form input material from a previous stage, e.g Input_A, which determine the columns j where values are to be entered. The emission data pⱼ or PCF pⱼ of the input material A, B, C, D, e.g. Input_A, are not known at this stage of processing and are the target to be deter-mined.

The input factor aᵢⱼ of the recipe may for example be taken from the RATIO column of the respective recipe. The contribution to the environmental property vector v may be the on-stage process emission contribution for the chemical process CP1-6 of a specific product, e.g. Output_E, which is recorded in row i of the output and is referred to as vᵢ. The on-stage emissions may be provided and may be mapped to the environmental property of the intermediate material, output material and/or end product. In an example, the on-stage emissions may be provided through measured data as provided by a measuring device associated with the chemical process.

In other words, the ratio aᵢⱼ may show the ratio of contribution for a provided input material A, B, C, D, Input_A, Input_B to the output product Output_E of the recipe. As the recipe may belong to a specific chemical process or production step CP1-6 the ratio aᵢⱼ may also be the ratio for the chemical process CP1-6. As at least one chemical process CP1-6 may be conducted in a process stage, aᵢⱼ may also influence the ratio of the process stage. The second case of adding ratio to the digital twin 1500 and/or combined recipe 1500 applies, when production network data are added to the combined matrix 1500. In that case the index i and/or row i of the matrix 1500 is an input material A, B, C, D, 200, Input_A and/or input material for a succeeding stage, and in particular of an intermediate E that forms the input to another process stage. In that case aᵢⱼ is a relative share “Rel. Share” 1402 of the interconnection ratio. The emission property that is to be determined, e.g. the PCF, pⱼ, is the PCF of a produced chemical Output_E or externally purchased chemical Ext_A, Ext_B. The values of the PCF, pⱼ, are to be determined and therefore may not be added to the matrix 1500. The emission property of that interconnection activity may be zero and the value to be added to the environmental property vector v for the transport ratio may be, vᵢ=0.

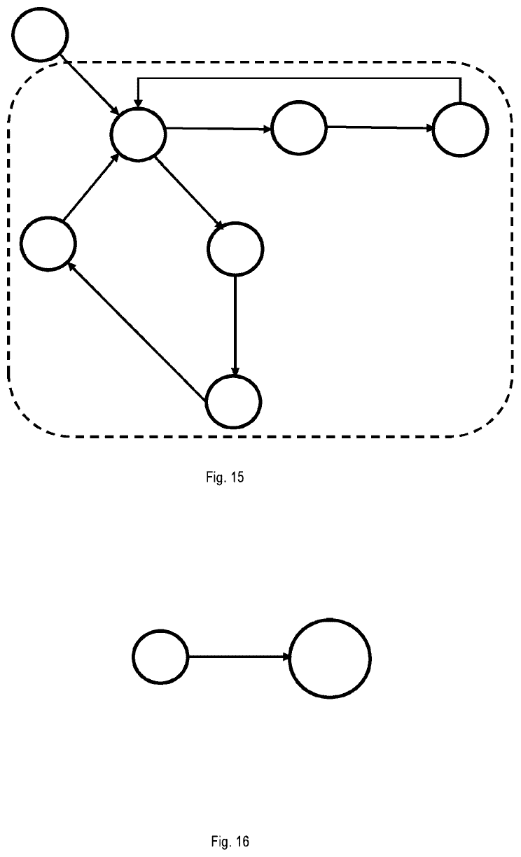

If in the third case an externally purchased product A, B, C, D, 200, Ext_A is to be added to the matrix 1500, the index i and/or row of the matrix 1500 is the purchased material A, B, C, D, 200, Ext_A and/or the externally purchased chemical A, B, C, D, 200, Ext_A, and the equation reduces to pᵢ=vᵢ, where vᵢ is the raw material PCF, i.e. only the environmental property vector v of the total emission properties gets an entry at the respective row i and/or index. In the example of FIG. 15 the value v=1 is added for Ext_A and the value 0.5 is added for Ext_B.

The dots in FIG. 15 indicate that the matrix is only a part for the situation shown in FIG. 14 and the dimension corresponds to the number of products and/or material that appear during a production process, i.e. the external purchased products A-D, the intermediates E-I that form the inputs to another stage and/or end products K-O.

In general, the column j of the matrix representation 1500 may relate to input material flows to chemical processes. The rows may relate to output material flows from chemical processes. In other words, the combined recipe 1500 is a representation of the chemical production network including all material flows that are connected to environmental contributions. Different indices may represent the same material either as an own production “OWN_PRODUCTION” within the sub-cluster or the chemical production network or as an external purchase “EXTERNAL-PURCHASE” to the chemical production network or the sub-cluster. In other words, the output of per chemical process may be the input to the sub-cluster reflected by the production network data and/or the input to another chemical process reflected by the chemical process recipe data. When all recipes and interconnection ratios are entered in the combined recipe matrix 1500 for every material used in the chemical processes and in particular for production one of the end products K-O, a row or line may be provided. Such line or row may be a sub-matrix per material. Every row of the matrix 1500 may hence correspond to the input-output model, however the assignment of the parameters and/or variables may be made dependent on the type of emission contribution to be allocated.

The combined recipe 1500 may provide a list which has for every output product K-O, that is provided at the system boundary, a link to the input material A-D that is received at the system boundary as a one-to-one relation in a condensed form. In the condensed form it is assumed that the intermediate stages are filtered out and may not be visible. In other words, for every input material A-D a line exists in the combined recipe 1500 that shows the relation between the input material A-D and the output product K-O substantially without indicating intermediates E-I. The consolidated form or condensed form may be reached by transforming the combined recipe 1500 and/or by accumulating columns and/or rows that may refer to intermediate process stages, e.g. to process stages that are not substantially directly connected with an input material A-D and/or product K-O. The combined recipe 1500 may thus consolidate emission contributions along the value chain.

Turning back to FIG. 12, to speed up the inversion and reduce the computing resources required for inversion of the attribution matrix graph data structures as for example described in the context of FIGS. 10 and 11 may be used. In particular, the directed graph data structure may be generated at least for part of the network-based representation that is associated with recirculation across chemical processes. This way the network-based representation may be efficiently transformed to the output material or product, e.g. end product, based representation. The network-based representation may be based on the attribution matrix as for example described in the context of FIGS. 12 to 13. The directed graph data structure may reflect the structure of the chemical production network. Chemical production networks may include material flows with cycles. Cycles may include material flows, in which output material is fed back into at least one preceding chemical process or any chemical process connected to at least one preceding chemical process. The preceding or prior chemical process may refer to any chemical process that produces output material which is directly or indirectly used to produce the input material of any subsequent chemical process. Cycles may relate to recirculation of output material of a first chemical process as input material to a second chemical process. The second chemical process may be a prior chemical process. One example of recirculation in chemical production networks is shown in FIG. 2 for output material 230 of chemical process 216, which is recircled to prior chemical pro-cess 204. In the graph data structure recirculation may be reflected by cycles as illustrated in FIG. 15.

The graph data structure may be transformed to combine cycles of the chemical production network. Such transformation of the graph data structure may be performed by graph instructions configured to transitively reduce vertices and edges forming cycles. FIG. 16 illustrates the transformed graph data structure of the chemical production network illustrated in FIG. 15. Here the cycles cumulate in one vertex. The cycles are reduced to a reduced vertex combining the cycles. The transformed, directed graph data structure may be topologically ordered. Such ordering of the graph data structure may be performed by graph instructions configured to topologically order vertices into a sequence, e.g. such that for every edge, the start vertex of the edge occurs earlier in the sequence than the ending vertex of the edge.

The ordered graph data structure may be used to generate the output material or product, e.g. end product, based representation of the chemical production network. For example, the cyclic parts of the network-based representation as generated by way of the attribution matrix according to the methods described in the context of FIGS. 13 and 14 may be transformed to a graph data structure, cycles may be eliminated through graph transformation and the result may be provided to the attribution matrix. The attribution matrix to be inverted may be generated based on a topologically ordered graph data structure. The data related to the vertices and edges of the graph data structure may be used to build a matrix structure including a block upper triangular matrix with block matrices based on the ordered graph structure. Such matrix structure may be inverted recursively. The recursive inversion may be determined efficiently to speed up compute time by inverting the diagonal block matrices and performing matrix multiplications for the non-diagonal block matrixes. In other embodiments the attribution matrix may be provided in graph data structure and the transformation to the end-product representation may be executed based on the graph data structure.

Based on the manipulation of the graph data structure and the inversion of the attribution matrix derivable from the transformed graph data structure, the network-based representation may be mapped to the product, e.g. end product, based representation. The product, e.g. end product, based representation of the chemical production network may be provided.

The transformed combined recipe or attribution matrix may be generated by matrix inversion. From the perspective of the end product the combined recipe 1500 gives a substantially full transparency by showing the input factors for substantially every upstream production or purchase. The transformed combined recipe or attribution matrix may provide factors relating the amount of an upstream product to be produced and/or generated for the production of a specific amount of end product. In an example, the combined recipe may indicate the quantity in kg of a certain upstream product that needs to be produced or purchased in order to produce one kg of the end product.

One row of the transformed combined recipe or attribution matrix (I-A)⁻¹ may represent one combined recipe for one product, in particular a matrix row may represent one transformed combined recipe. Each entry in the trans-formed combined recipe (I-A)⁻¹ may define the factor which is to be applied by a multiplication to the corresponding v-entry in the environmental property vector v on stage level and/or raw material level in order to get the vector of environmental contributions on the end product level. Each row of the transformed combined recipe may be seen as a consolidated recipe for the end product. In other words, the environmental property vector v on the on-stage level may comprise the on-stage emissions and raw material PCFs. Consequently, each entry of the inverse matrix (I-A)⁻¹ may represent the amount of a raw material production and/or of an upstream production that is needed to produce one kg of the end product.

Even though the attribution matrix will become huge for large value chains and/or production paths, calculating and storing transformed combined recipe matrix (I-A)⁻¹ and thus the consolidated recipes in it may be useful, to simplify the PCF monitoring by increasing the transparency.

The calculation of the transformed combined recipe or attribution matrix (I-A)⁻¹ may take a couple of hours even on a high-performance computing infrastructure. The time may increase exponentially with increasing value chains. As long as the basic recipes and/or the interconnection ratios do not change, changes in the on-stage emissions v and/or raw material emissions v may be easily taken into account, since they do not affect the transformed input factor matrix for determining the product related emission data pᵢ. Since a matrix inversion may be time-consuming for large value chains the calculation of the transformed combined recipe matrix (I-A)⁻¹ may become complex and impractical. A large chemical company may have more than ten thousand individual products each having an individual consolidated recipe. In such a case the calculation of all individual consolidated recipes may be time consuming. In order to speed-up the calculation, in general the inversion of large matrices may be avoided. The combined recipe matrix (I-A) that may be generated for a production network of the chemical industry may be a sparse matrix with only view entries. This matrix structure may be used as a basis for to speed up the inversion of (I-A).

In an example graph theory is used to reorder the matrix (I-A). In one example the graph theory may be used for reordering a matrix for the calculation of a product related environmental property, e.g. of a PCF value. In a further example the graph theory may be used for a matrix inversion of a matrix with 1 to 1,000 rows, in another example the graph theory may be used for a matrix inversion of a matrix with 100 to 10,000 rows, in yet another example the graph theory may be used for a matrix inversion of a matrix with 10,000 to 60,000 entries.

The method for reordering the matrix (I-A) by using graph theory may comprise creating a graph representation of the value chain and/or production path. Strongly connected components may be identifies, wherein a strongly connected component (SCC) is a subgraph, where directed paths exist between every pair of nodes in each direction. These strongly connected components may correspond to cycles of recirculation in the value chain. These cycles or recirculations may be differentiated from loops or forks in the recipes.

The method may further comprise shrinking of each identified SCC to a single combined node in order to generate an acyclic graph. The single combined node may substantially have incoming and outgoing edges but prevents feed-back edges. By forming a single combined node the cycles may not be cancelled and/or eliminated but just algorithmically combined. It may still be accepted that the overall supply chain and/or the overall production path has cycles.

The method may provide for computing a topological sorting for the resulting graph. A topological sorting is an ordering of the graph's nodes or entries, for example the ratios aᵢⱼ of the process data such that all edges point from a node aᵢⱼ with a lower to a node with a higher index. It may be proven that a topological sorting exists for every acyclic graph.

The result of the graph transformation may be written back to the matrix representation. A block upper triangular matrix may be generated by ordering and/or sorting the matrix by the indices i, j of the topological sorting and aligning the columns, which correspond to an SCC, in an arbitrary order next to each other:

$$( \begin{matrix}{M}_{11} & {M}_{12} & {M}_{13} & … & {M}_{1 t} \\ 0 & {M}_{22} & {M}_{23} & … & {M}_{2 t} \\ 0 & 0 & {M}_{33} & … & {M}_{3 t} \\ … & … & … & … & … \\ 0 & 0 & 0 & … & {M}_{tt}\end{matrix} )$$

A further stage of the method may provide for computing the inverse of the matrix recursively. Assumed that a sub-matrix has the following substructure

$$( \begin{matrix}A & B \\ 0 & D\end{matrix} ) ,$$

it may be checked whether A and D are invertible. If A and D are invertible, then the inverse of

$$( \begin{matrix}A & B \\ 0 & D\end{matrix} )$$

is

$$( \begin{matrix}{A}^{- 1} & - {A}^{- 1} {BD}^{- 1} \\ 0 & {D}^{- 1}\end{matrix} ) .$$

A, B, D may be used for showing the general structure of the patterns that are checked and may correspond to a subgroup of M₁₁, . . . , Mₜₜ. The recursive application may result in the effect that only the block matrices M₁₁, . . . , Mₜₜ on the diagonal of the matrix need to be inverted and the rest of the inverse can be calculated via simple matrix multiplications which do not consume too much calculation power.

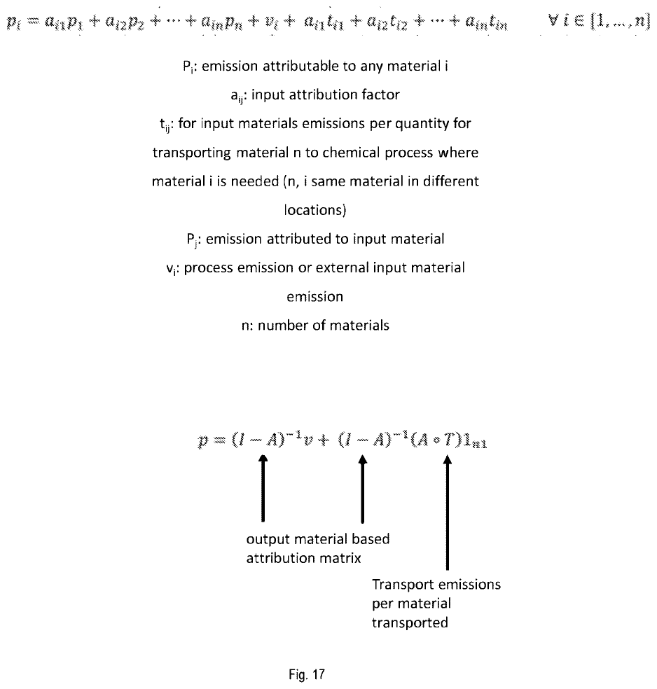

FIG. 17 illustrates another example of the model representation for emission contributions associated with the chemical production network.

Transport emissions may relate to material being transported within the chemical production network boundary. Such emissions may relate to Scope 1 or 3 emissions, that may be taken into account next to emissions related to the chemical process and/or to the input materials provided to the chemical production network. Equ. 1 of FIG. 17 illustrates one possible extension of the model illustrated in FIG. 12. In the scenarios lined out in the context of FIG. 12 the transport emissions may be relevant to input materials provided between subclusters and/or plants. Transforming Equ. 1 in line with the example of FIG. 12 provides for the inverted attribution factor matrix illustrated. The inverse attribution factor matrix may represent the amount of intermediate material needed to produce one kilogram of end product. As lined out in FIG. 12 aij for the input material may relate to the consumption share for each input material. Hence the product provides the quantity of intermediate material that needs to be transported to produce one kilogram of end-product. tᵢⱼ may relate to the emissions per kilogram for transporting product i to the production site and/or to the process where product i is being needed. For example, intermediate E in FIG. 13. The indexes i and j represent the same material, e.g. intermediate, at different locations. This extension with transport emissions may be considered in the case where i is any input material. For example, for rows i in FIG. 13 with an input product may be extended to transports. The equation p=Ap+v remains substantially unchanged in all other cases. In the com-pact form of the equation for considering transport emissions 1ₙ₁ is a vector of ones of length n. T is a matrix comprising the transport emissions tᵢⱼ.

Each multiplication product

$${( I - A )}_{ij}^{- 1} {a}_{ji}$$

of the term (I-A)⁻¹(A·T)1ₙ₁ may relate to the amount of any material, e.g. intermediate, that needs to be transported from the source to the intermediate production step to produce 1 kg of the end product. Each row in the transformed combined recipe matrix (I-A)⁻¹ may represent one product, in particular one chemical end-product. In this equation the factors aⱼᵢ may represent an interconnection ratio. The term

$${( I - A )}_{ij}^{- 1}$$

is a representation of the amount of an intermediate output material that is needed as input material in another stage downstream in order to produce 1 kg of the end product. The end product may be produced one or more stage(s) away in the downstream direction. The multiplication of the amount of intermediate

$${( I - A )}_{ij}^{- 1}$$

needed at the actual stage for 1 kg of the end-product with the interconnection ratio entry aⱼᵢ then generates the actually transported amount of that input material from one of the sources to the downstream production facility.

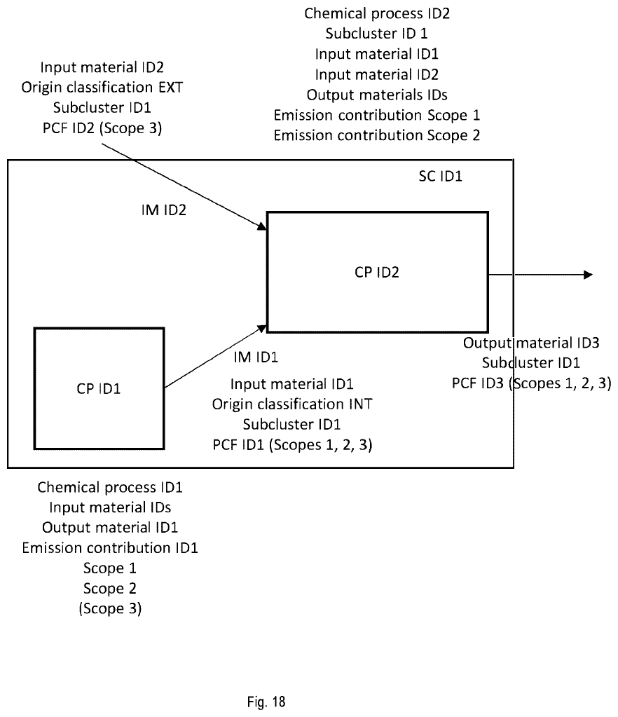

FIG. 18 illustrates another example extract of the digital representation including emission contributions.

FIG. 18 corresponds to FIG. 9 from the general line out. In contrast to FIG. 9 the data is further enriched with emission contributions as provided by the network-based and/or output material, e.g. end-product, based data structure. In such data structure the inverted attribution matrix as for example described in the context of FIGS. 12-17 provides the emission contribution factor for the amount of externally purchased or upstream production needed to produce one kilo gram of end product. Applying these emission contribution factors to the emission contribution associated with externally purchased input materials and/or associated with the chemical process, hence leads to accumulated emission contributions per input material externally purchased and/or chemical process required to produce the end product. The emission contribution associated with the chemical process may include the emission contribution based on the share of input materials used for the chemical process and/or the emission contribution based on the share of direct and/or energy emissions associated with the chemical process. The different emission contributions may be tagged as Scope 1, 2 or 3 emissions. In the example of FIG. 18, the output material with ID3 may have a PCF ID3. The PCF ID3 may be composed of the PCF ID1 and the PCF ID2 according to the emission contributions based on the share of input materials used for the chemical process. The PCF ID1 may include the emission contributions from chemical process ID1. Hence the different emission contributions may be mapped according to the end-product logic based on the input material ID2 provided to the chemical production network and the upstream production CP ID1 needed to produce output material ID3. For the upstream production CP ID1, the emission contribution may be mapped on output material level of the chemical process CP ID1 or equivalently to the input material level of chemical process CP ID2. The end-product based mapping hence gives full transparency on how the product carbon footprint of output material ID3 is composed per chemical process preceding the final chemical process CP ID2 producing output material ID3. In other words, via the inverse attribution matrix, the raw material input or production quantity of the preceding chemical production process may be related to the raw material PCF or the chemical process PCF of the preceding chemical process. The PCF contributions to the output material exiting the chemical production network may hence be determined based on the PCF contribution of each preceding chemical process of the chemical production network leading to the end product.

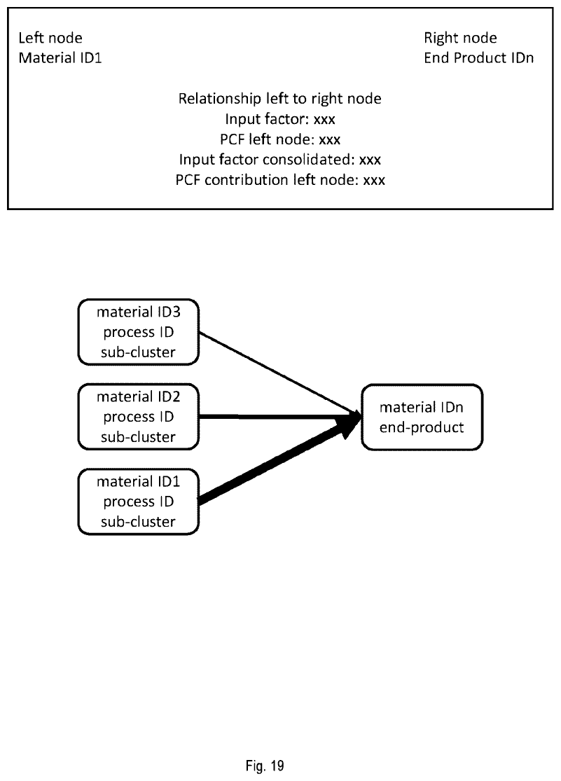

By transforming the network-based representation to the end-product based representation as discussed above, may provide the quantity of input material and upstream production required to produce the output material that exists the chemical production network. In other words, rather than solving the matrix equation p=Ap+v or (I-A) p=v with p signifying the PCF contribution from all input materials and v signifying the PCF contribution from external input materials and process contributions, the matrix equation p=(I-A)⁻¹ v may be solved. The input factor matrix A may hence transform from mapping the quantity of input material or input material share per produced material to PCF contribution per process or external input materials, to mapping the quantity of input material and upstream production contributing to the total PCF per produced material. In yet other words, the network-based representation of the chemical production network following the network logic from output material exiting the chemical production via intermediate input materials to input materials may be transformed to the end-product based representation following the output material logic including input materials and chemical processes required to produce the output material. Advantageously the latter representation is easier to interpret and allows for more reliable monitoring. The determination of the end-product based representation may be performed as described in the context of FIGS. 12-16. FIG. 19 illustrates an example user interface generated based on the network-based and/or product-based representation.

The network-based or product-based representation, as for instance generated based on production data and production network data, may be transformed to a graph data structure. Graph data structures may include vertices (or nodes) and edges connecting the vertices. The vertices may relate to chemical processes, subclusters and/or the materials. The edges may relate to material flow. The vertices and/or edges may be associated with metadata specifying properties of the vertices and/or edges. The vertices may specify a relationship between vertex pairs and an orientation. The graph data structure may be a directed graph structure. The graph data structure may be built as for example described in the context of FIGS. 10, 11, 15, 16, 18 and related disclosure.

FIG. 19 illustrates a user interface generated based on such graph data structure. The user interface illustrates the node for the end product with material IDn as right node. The user interface illustrates three example nodes for the preceding chemical processes producing material ID1, ID2, ID3 as input materials for the right node as left nodes. The arrows between the nodes signify material flow from the preceding nodes with material ID1, ID2, ID3 to the following node with material IDn. The weight of the arrows signifies the PCF contribution of the input materials produced in the preceding chemical processes to the end product. The user interface may be an interactive user interface with a pop up appearing on user interaction, e.g. mouse over. In the example the pop up on the top illustrates the details of the arrow pointing from material ID1 to end product IDn.

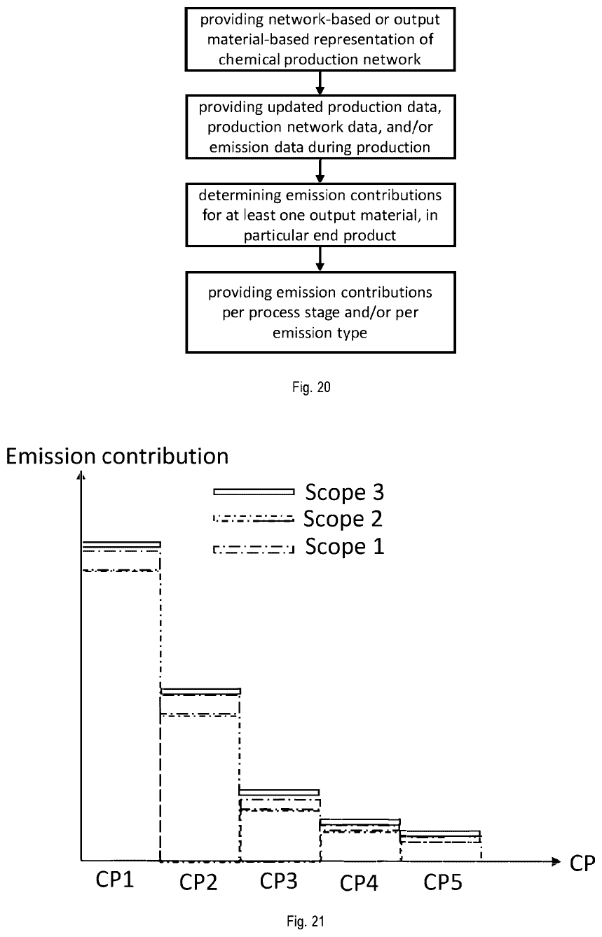

FIG. 20 illustrates an example of a flowchart for monitoring resource contributions such as emission contributions of chemical production network per network component or per end product production path or chain of the chemical production network.

For monitoring resource contributions such as emission contributions the network-based and/or product-based representation of chemical production network may be provided. The network-based and/or product-based representation may be generated as for example described in the context of FIGS. 5-17.

The production data, production network data, and/or resource usage data may be updated e.g. on or during production in real-time or over a time period. The production data, production network data, and/or resource usage data may be gathered and processed as for example described in the context of FIGS. 5-9.

The resource contributions such as the emission contributions for at least one end product or for at least one network component, such as the chemical process, may be determined. Here the contributions may be determined as for example described in the context of FIGS. 5, 12-17.

The emission contributions per end product, per network component, per process stage and/or per resource usage type, such as emission type, may be provided.

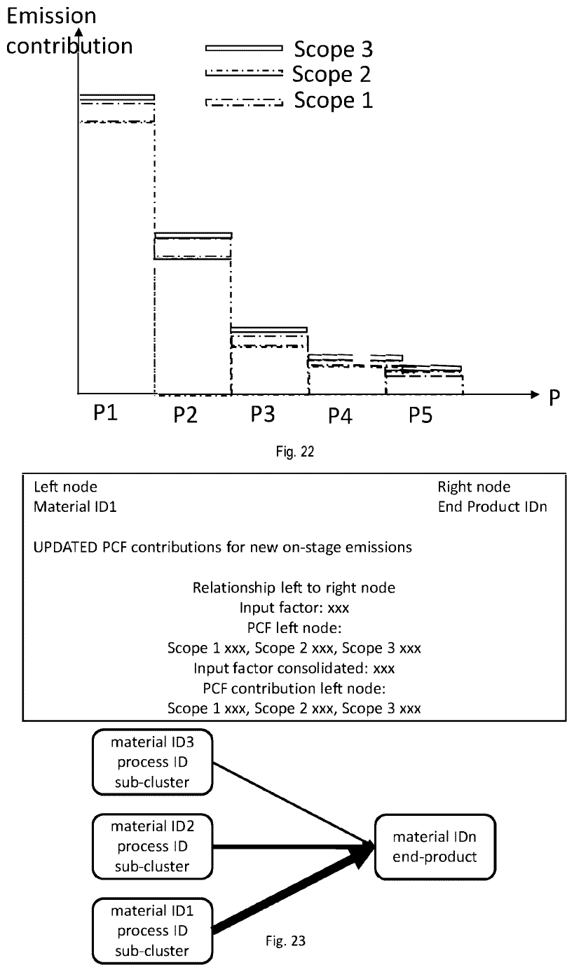

The results of the methods disclosed herein are illustrated via a visualization that may be provided to a user interface. FIG. 21 illustrates a schematic example of emission contributions per chemical process. FIG. 22 illustrates a schematic example of emission contributions per output material.

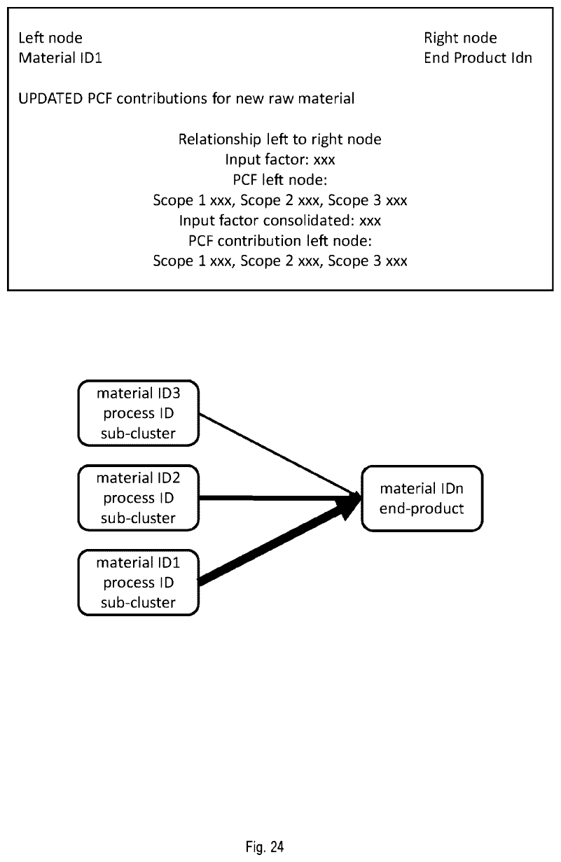

FIGS. 23-26 illustrate schematic examples of emission contributions in a value chain of the chemical production network. FIGS. 23-26 illustrate example user interfaces generated based on the network-based representation and the product-based representation for updated on stage data. FIGS. 24-26 illustrate schematic examples of monitoring emission contributions in a value chain of the chemical production network based on raw materials, process emissions and/or production volumes.

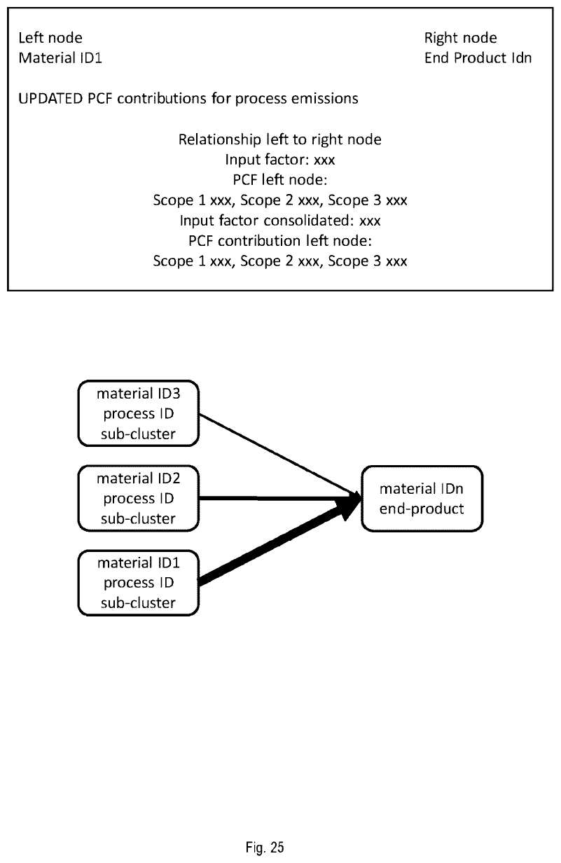

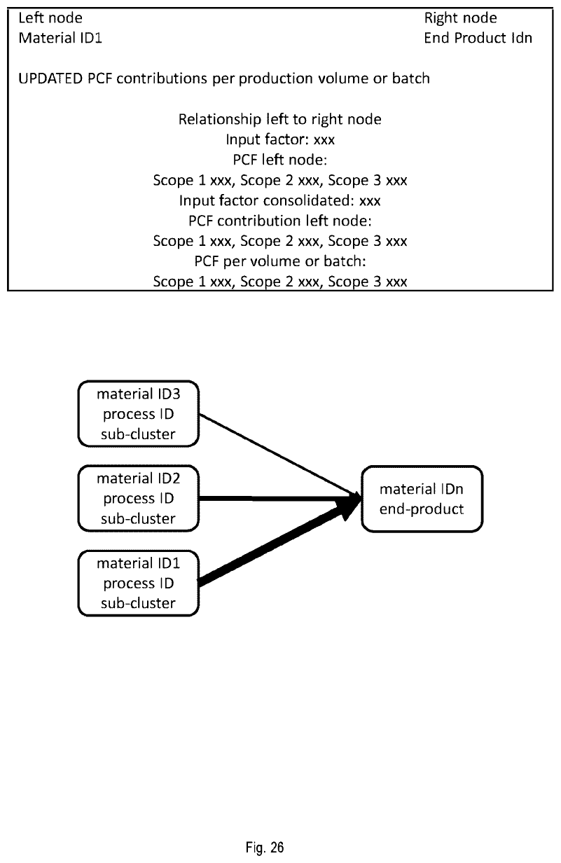

The user interface of FIG. 23 illustrates the node for the end product with material IDn as right node. The user interface illustrates three example nodes for the preceding chemical processes with material ID1, ID2, ID3 as left nodes. The arrows between the nodes signify material flow from the preceding nodes with material ID1, ID2, ID3 to the following node with material IDn. The weight of the arrows signifies the PCF contribution of the preceding input materials produced by chemical processes to the end product. In the example, the pop up on the top illustrates the details of the arrow pointing from material ID1 to end product IDn. The vertices may be run through in sequential order. E.g. upon user interaction the display may shift the vertices to the right or left based on the graph data structure. In FIG. 23 the PCF contribution to on-stage emissions are updated, e.g. due to more green energy being used than in the former run. In FIG. 24 the PCF contribution of raw materials provided to the chemical production network are updated, e.g. due to raw material with lower PCF being available. In FIG. 25 the PCF contribution to process emissions are updated, e.g. due to changes in the chemical process setup that reduce PCF. In FIG. 26 the PCF contribution per production volume or batch are updated, e.g. due to change in production volumes or batches.

Other examples of user interfaces may be adaptive with regard to the displayed vertices, edge(s) and metadata displayed. Adaption may be triggered upon user interaction. For example, the vertices and/or edge(s) may be expanded or collapse to provide more or less vertices and/or edge(s) based on the graph data structure. Further for example, multiple vertices may be collapsed to a summed vertex or a summed vertex may be expanded to multiple vertices including at least one edge between the vertices. E.g. chemical process vertices with associated material flow edges may by collapsed to subcluster vertex with associated material flow edges or a subcluster vertex may be expanded to chemical process vertices with associated material flow edges. This way the mapping of the environmental property contributions of the chemical production infrastructure to the environmental property of the output materials, e.g. end products, or network components can be simplified for enhanced monitoring.

The present disclosure has been described in conjunction with preferred embodiments as examples as well. However, other variations can be understood and effected by those persons skilled in the art and practicing the claimed invention, from the studies of the drawings, this disclosure and the claims. Notably, in particular, the any steps presented can be performed in any order, i.e. the present invention is not limited to a specific order of these steps. Moreover, it is also not re-quired that the different steps are performed at a certain place or at one node of a distributed system, i.e. each of the steps may be performed at a different nodes using different equipment/data processing units.

In the claims as well as in the description the word “comprising” does not exclude other elements or steps and the indefinite article “a” or “an” does not exclude a plurality. “can” or “may” refers to optional features. A single element or other unit may fulfill the functions of several entities or items recited in the claims. The mere fact that certain measures are recited in the mutual different dependent claims does not indicate that a combination of these measures cannot be used in an advantageous implementation.

The present disclosure has been described in conjunction with preferred embodiments and examples as well. However, other variations can be understood and effected by those persons skilled in the art and practicing the claimed invention, from the studies of the drawings, this dis-closure and the claims.

Any steps presented herein can be performed in any order. The methods disclosed herein are not limited to a specific order of these steps. It is also not required that the different steps are per-formed at a certain place or in a certain computing node of a distributed system, i.e. each of the steps may be performed at different computing nodes using different equipment/data processing.

Providing in the scope of this disclosure may include any interface configured to provide data. This may include an application programming interface, a human-machine interface such as a display and/or a software module interface. Providing may include communication of data or submission of data to the interface.

As used herein “determining” also includes “initiating or causing to determine”, “generating” also includes “initiating and/or causing to generate” and “providing” also includes “initiating or causing to determine, generate, select, send and/or receive”. “Initiating or causing to perform an action” includes any processing signal that triggers a computing node or device to perform the respective action.

In the claims as well as in the description the word “comprising” does not exclude other elements or steps and the indefinite article “a” or “an” does not exclude a plurality. A single element or other unit may fulfill the functions of several entities or items recited in the claims. The mere fact that certain measures are recited in the mutual different dependent claims does not indicate that a combination of these measures cannot be used in an advantageous implementation.

All terms and definitions used herein are understood broadly and have their general meaning.

1. A method for monitoring an environmental property of an output material produced by a chemical production network, wherein the chemical production network includes multiple interconnected chemical processes for producing one or more output material(s) from one or more input material(s), the method comprising:
providing a product-based representation of the chemical production network adapted for allocating one or more environmental property contribution(s) associated with the production of one or more output material(s) through multiple interconnected chemical processes to the environmental property contributions per output material(s);
providing one or more environmental property contribution(s) related to the production of one or more output material(s) through multiple interconnected chemical processes;
generating the environmental property contributions per output material(s) by mapping the one or more environmental property contribution(s) related to the production of one or more output material(s) through multiple interconnected chemical processes according to the product-based representation of the chemical production network; and
providing the environmental property contributions to the output material.

2. The method of claim 1, wherein providing a product-based representation of the chemical production network includes
gathering production data associated with the chemical processes and material flows from input material(s) to output material(s) per chemical process,
gathering production network data associated with material flows to the chemical production network and/or between chemical processes of the chemical production network, and
generating a product-based representation of the chemical production network based on the production data and the production network data.

3. The method of claim 1, wherein generating a product-based representation includes generating a network-based representation that maps the chemical production network based on the production data and the production network data to one or more material input-output relations per chemical process, transforming the network-based representation to a product-based representation by determining output material relations from the input-output relations for mapping the environmental property contributions from the production of one or more output material(s) to the environmental property contributions per output material(s).

4. The method of claim 3, wherein the network-based representation is adapted for allocating one or more environmental property contribution(s) associated with the production of one or more output material(s) according to a network flow logic, wherein the product-based representation is adapted for allocating one or more environmental property contribution(s) associated with the production of one or more output material(s) according to a material use logic.

5. The method of claim 1, wherein at least part of the product-based representation is transformed to a graph data structure associated with chemical processes or subclusters as vertices and material flows as edges, wherein the graph data structure includes metadata associated with the environmental property contributions per output material production path, wherein the environmental property contributions per chemical process or subcluster are added to the vertices and the emission contributions per material flow are added to the edges.

6. The method of claim 1, wherein a network-based representation of the chemical production network is provided, wherein at least part of the network-based representation is transformed to a graph data structure associated with chemical processes or subclusters as vertices and material flows as edges, wherein the graph data structure includes metadata associated with the environmental property contributions per network component, wherein the environmental property contributions per chemical process or subcluster are added to the vertices and the emission contributions per material flow are added to the edges.

7. The method of claim 1, wherein the one or more environmental property contribution(s) relate at least to the one or more input material(s) provided to the chemical production network, the chemical processes of the chemical production network, and/or transport of input material(s) inside the chemical production network.

8. The method of claim 1, wherein the one or more environmental property contribution(s) associated with the production of one or more output material(s) are provided per input material, per chemical process and/or per transport of input material(s) inside the chemical production network.

9. The method of claim 1, wherein the environmental property contributions relate to carbon emissions of the chemical production network, wherein the environmental property contributions relate at least to carbon emissions associated with the one or more input material(s) provided to the chemical production network, carbon emissions associated with the chemical processes of the chemical production network, carbon emissions associated with the transport of input material(s), and/or carbon emissions associated with energy usage of the chemical production network.

10. The method of claim 1, wherein providing one or more environmental property contribution(s) related to the production of one or more output material(s) includes gathering emission data from distributed monitoring systems of the chemical production network, wherein the emission data relates to emissions generated on production of output materials by the chemical production network.

11. The method of claim 10, wherein the emission data is provided per chemical process and/or per subcluster including one or more chemical process(es).

12. The method of claim 10, wherein the emission data is at least partially provided per subcluster including one or more chemical process(es), wherein the emission data is at least partially allocated to chemical process(es) of the subcluster.

13. An apparatus for monitoring an environmental property of an output material produced by a chemical production network, wherein the chemical production network includes multiple chemical processes for producing one or more output material(s) from one or more input material(s), the apparatus comprising:
a provisioning interface configured to provide a product-based representation of the chemical production network adapted for allocating one or more environmental property contribution(s) associated with the production of one or more output material(s) through multiple interconnected chemical processes to the environmental property contributions per output material(s) and to provide one or more environmental property contribution(s) related to the production of one or more output material(s) through multiple interconnected chemical processes;
a contributions generator configured to generate the environmental property contributions per output material(s) by mapping the one or more environmental property contribution(s) related to the production of one or more output material(s) through multiple interconnected chemical processes according to the product-based representation of the chemical production network; and
a data provider configured to provide the environmental property contributions to the output material.

14. An output material produced by a chemical production network, wherein an environmental property of the output material is provided according to the method of claim 1.

15. A method for determining an environmental property of a product produced from output material comprising using the environmental property of the output material provided by the method of claim 1.

16. An output material produced by a chemical production network, wherein an environmental property of the output material is provided by the apparatus of claim 13.

17. A method for determining an environmental property of a product produced from output material comprising using the environmental property of the output material provided by the apparatus of claim 13.
